(19) United States
(12) Patent Application Publication
(10) Pub. No.: US 2026/0027210 A1
(43) Pub. Date: Jan. 29, 2026

(54) THERAPEUTIC COMBINATIONS OF TITR EFFECTORS WITH RADIATION THERAPY

(71) Applicant: Varian Medical Systems, Inc., Palo Alto, CA (US)

(72) Inventors: Sophia PFISTER, Redwood City, CA (US); Marta VILALTA, Palo Alto, CA (US); Ricky Anupam SHARMA, Palo Alto, CA (US); Yurun ZHANG, Sunnyvale, CA (US); Sandra HATCHER, Pacifica, CA (US); Vikash BHAGWANDIN, San Mateo, CA (US); Ziyun DING, Redwood City, CA (US)

(21) Appl. No.: 18/998,197

(22) PCT Filed: Jul. 27, 2023
(86) PCT No.: PCT/US2023/071112

§ 371 Date: Jan. 24, 2025

Related U.S. Application Data
(60) Provisional application No. 63/392,759, filed on Jul. 27, 2022.

(51) Int. Cl. A61K 41/00; C07K 16/28

(52) U.S. Cl. CPC A61K 41/0038; C07K 16/2815; C07K 16/2818; C07K 16/2866

(57) ABSTRACT

This disclosure relates to therapeutic combinations of TITR effectors such as CCR8 targeting agents, radiation therapy and checkpoint inhibitors and methods of using the same for the treatment of cancer. The disclosure also relates to methods of stratifying a patient population for treating with the therapeutic combination.

### RELATED APPLICATIONS

This application claims priority to, and the benefit of, U.S. Provisional Application 63,392,759, filed Jul. 27, 2022, the entire contents of which are incorporated herein by reference.

### TECHNICAL FIELD

This disclosure relates to therapeutic combinations of tumor infiltrating regulatory T cell (TITR) effectors with radiation therapy and methods of using the same for the treatment of cancer.

### BACKGROUND

Tumor infiltrating regulatory T cells (TITRs) originate from resident tissue lymphoid progenitors, but differentiate into a unique Treg subpopulation during tumorigenesis, defined by a specific gene expression signature, which suppress the ability of TILs or CD8+ cytotoxic T cells to kill tumor cells. Thus, the TITR population is a unique cell population that is part of the Treg subfamily of CD4+/CD25+/FoxP3+ T cells. In contrast, Tregs of the blood or normal tissue type regulate autoimmunity and infections, which are defined by a different gene expression signature.

Radiation therapy induced immunogenic cell death, that is intended to mount an effective cytotoxic T cell response, is suppressed by tumor infiltrating Tregs (TITRs). Targeting pan-Treg has been reported to augment autoimmune disease and increased susceptibility to infectious diseases.

### SUMMARY

In one aspect, provided herein is a method of targeting tumor infiltrating regulatory T cell (TITR) cells in a subject, the method comprising administering to the subject a TITR effector and a radiation therapy. In some embodiments, provided herein is a method of targeting tumor infiltrating regulatory T (TITR) cells in a subject, the method comprising administering to the subject a TITR effector and a radiation therapy. In some embodiments, the TITR effector is a CCR8 targeting agent. In some embodiments, the CCR8 targeting agent is an anti-CCR8 antibody. In some embodiments, the TITR effector and the radiation therapy are administered concurrently. In some embodiments, the TITR effector and the radiation therapy are administered sequentially. In some embodiments, the TITR effector, the radiation therapy, or both are administered at least one time, at least two times, at least three times, at least four times or at least five times. In some embodiments, the subject is afflicted with cancer. In some embodiments, the cancer is breast cancer, lung cancer, head and neck cancer, liver cancer, pancreatic cancer, brain cancer, colorectal cancer, or prostate cancer. In some embodiments, the cancer has previously been treated with radiation therapy and/or the TITR effector. In some embodiments, the cancer is resistant to radiation therapy and/or the TITR effector. In some embodiments, the radiation therapy is electron radiation, proton radiation, photon radiation, a radiopharmaceutical, or brachytherapy, In some embodiments, the radiation therapy is ultra-high dose rate (FLASH) radiation therapy.

In some embodiments, the method results in a reduction in TITR number in the cancer relative to the TITR number prior to the administration. In some embodiments, the method results in a decrease of about 5%, about 10%, about 20%, about 30%, about 40%, about 50%, about 60%, about 70%, about 80%, or about 90% of the number of TITR cells in the subject relative to the number of TITR cells in the subject prior to the administration of the TTR effector and the radiation therapy.

In another aspect, provided herein is a method of treating cancer in a subject, the method comprising (a) obtaining a tumorsample from the cancer and a normal tissue sample; (b) quantifying the expression level of at least 3, at least 4, at least 5, at least 6, at least 7, at least 8, at least 9, at least 10, at least 11, at least 12, at least 13, at least 14, at least 15, at least 16, at least 17, at least 18, at least 19, at least 20 genes, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110 genes, at least 115 genes, at least 120 genes, at least 125, at least 130 genes, at least 135 genes, at least 140 genes, at least 145, at least 150 genes, at least 155 genes, at least 160 genes, at least 165, at least 170 genes, at least 175 genes, or at least 180 genes and (c) administering to the subject a combination therapy comprising a TITR effector and a radiation therapy if a TITR gene signature is identified in the tumor sample compared to the normal tissue sample using an over-representation/enrichment method; wherein the genes are selected from CCR8, CD80, CSF2RB, ENTPD1, EBI3, TNFRSF8, TNFRSF18, DUSP4, SDC4, LAPTM4B, TNFRSF4, SEC14L1, TNFRSF9, IRAK2, FNDC3B, IKZF4, IL1R2, NINJ1, SYNGR2, TNIP2, SSH1, ARHGEF12, GCNT1, NCF4, MAPKAPK2, RHBDD2, TRAF3, IRF5, ACOT9, HIVEP3, RFK, UEVLD, BCL2L1, FAM129A, BATF, RHOC, ZNRF1, PMAIP1, PLAGL2, MRPS6, IL1RL1, CREB3L2, CPD, PICALM, CCR5, CTNNA1, GALC, TTC39C, CYFIP1, STAT1, FURIN, TRAF1, GBP5, HIF1A, UBE2L6, PTPRJ, TNFRSF1B, REM, CAPN2, TRIM16, CD74, MAP2K3, TIGIT, CTSH, TAP1, SKAP2, ITGAE, JAK3, CAPG, OPTN, NDRG1, DUSP16, CD274, ICOS, GLRX, SAMSN1, ANXA4, MAPKAPK3, KATNAL1, CDKN1A, SNX9. PFKL, ABI2, ATP2B4, GM2A, PGM2, RIPK3, ISGJ5, NAMPT, MVP, GOT1, MXD1, TMBIM1, RORA, PCYT1A, N4BP1, LAMP2, TMEM159, OSBPL3, VIM, ATF3, CXCR3, ALDOC, PLP2, FAM126A, OXSR1, GRN, APAF1, ANK3, ANKRD55, ANXA2R, ATM, BAZ2B, CCR7, CD200, CECR1, CLUHP3, CXCR5, DPH5, EPHA1, EPHA4, FAM174B, FAM65B, FASTKD2, FOXP1, GIMAP4, GIMAP5, GIMAP6, GIMAP7, GIMAP8, GZMK, HIVEP2, IL10, INADL, INPP4B, IQGAP2, KCNA3, KIAA1009, KIAA2026, KLF3, KLRG1, LDLRAP1, LEF1, LMO7, LOC100130231, LPAR6, LRIG1, LY9, MAP3K3, MS4A1, NAP1L2, OBSCN, PAR-SN, PLAC8, PLAG1, PLEK, PLK2, PLXDC1, PRKCQ-AS1, RNF214, SERINC5, SESN1, SLAMF7, SLC16A7, SLC9A6, SMAD3, SPINT2, SSBP2, STXBP5, TCF7, TENM1, TMEM243, TRAT1, TTC21B, TXK, ZBTB10, ZNF248, ZNF480, ZNF737, ZNF780B, and ZNF91.

In another aspect, provided herein is a method for selecting a subject having a cancer for treatment with a combination therapy comprising a TITR effector and a radiation therapy, the method comprising (a) obtaining a tumor sample from the cancer and a normal tissue sample; (b) quantifying the expression level of at least 3, at least 4, at least 5, at least 6, at least 7, at least 8, at least 9, at least 10, at least 11, at least 12, at least 13, at least 14, at least 15, at least 16, at least 17, at least 18, at least 19, at least 20 genes, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110 genes, at least 115 genes, at least 120 genes, at least 125, at least 130 genes, at least 135 genes, at least 140 genes, at least 145, at least 150 genes, at least 155 genes, at least 160 genes, at least 165, at least 170 genes, at least 175 genes, or at least 180 genes; and (c) selecting the subject for treatment if a TITR gene signature is identified in the tumor sample compared to the normal tissue sample using an over-representation/enrichment method; wherein the genes are selected from CCR8, CD80, CSF2RB, ENTPD1, EBI3, TNFRSF8, TNFRSF8, DUSP4, SDC4, LAPTM4B, TNFRSF4, SEC14L1, TNFRSF9, IRAK2, FNDC3B, IKZF4, IL1R2, NINJ1, SYNGR2, TNIP2, SSH1, ARHGEF2, GCNT1, NCF4, MAPKAPK2, RHBDD2, TRAF3, IRF5, ACOT9, HIVEP3, RFK, UEVLD, BCL2L1, FAM129A, BATF, RHOC, ZNRF1, PMAIP1, PLAGL2, MRPS6, IL1RL1, CREB3L2, CPD, PICALM, CCR5, CTNNA1, GALC, TTC39C, CYFIP1, STAT1, FURIN, TRAF1, GBP5, HIF1A, UBE2L6, PTPRJ, TNFRSF1B, REM, CAPN2, TRIM16, CD74, MAP2K3, TIGIT, CTSH, TAP1, SKAP2, ITGAE, JAK3, CAPG, OPTN, NDRG1, DUSP16, CD274, ICOS, GLRX, SAMSN1, ANXA4, MAPKAPK3, KATNAL1, CDKN1A, SNX9. PFKL, ABI2, ATP2B4, GM2A, PGM2, RIPK3, ISG15, NAMPT, MVP, GOT1, MXD1, TMBIM1, RORA, PCYT1A, N4BP1, LAMP2, TMEM159, OSBPL3, VIM, ATF3, CXCR3, ALDOC, PLP2, FAM126A, OXSR1, GRN, APAF1, ANK3, ANKRD55, ANXA2R, ATM, BAZ2B, CCR7, CD200, CECR1, CLUHP3, CXCR5, DPH5, EPHA1, EPHA4, FAM174B, FAM65B, FASTKD2, FOXP1, GIMAP4, GIMAP5, GIMAP6, GIMAP7, GIMAP8, GZMK, HIVEP2, IL10, INADL, INPP4B, IQGAP2, KCNA3, KIAA1009, KIAA2026, KLF3, KLRG1, LDLRAP1, LEF1, LMO7, LOC100130231, LPAR6, LRIG1, LY9, MAP3K3, MS4A1, NAP1L2, OBSCN, PAR-SN, PLAC8, PLAG1, PLEK, PLK2, PLXDC1, PRKCQ-AS1, RNF214, SERINC5, SESN1, SLAMF7, SLC16A7, SLC9A6, SMAD3, SPINT2, SSBP2, STXBP5, TCF7, TENM1, TMEM243, TRAT1, TTC21B, TXK, ZBTB10, ZNF248, ZNF480, ZNF737, ZNF780B, and ZNF91. Steps (b) and (c) can be performed on the samples when the samples are outside of the body of a subject.

In some embodiments, the TITR effector is a CCR8 targeting agent. In some embodiments, the CCR8 targeting agent is an anti-CCR8 antibody. In some embodiments, the radiation therapy is electron radiation, proton radiation, or photon radiation. In some embodiments, the radiation therapy is ultra-high dose rate (FLASH) radiation therapy. In some embodiments, the cancer is breast cancer, lung cancer, head and neck cancer, liver cancer, pancreatic cancer, brain cancer, colorectal cancer, or prostate cancer.

In another aspect, provided herein is the use of a plurality of agents specific to quantifying the expression level of at least 3, at least 4, at least 5, at least 6, at least 7, at least 8, at least 9, at least 10, at least 11, at least 12, at least 13, at least 14, at least 15, at least 16, at least 17, at least 18, at least 19, at least 20 genes, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110 genes, at least 115 genes, at least 120 genes, at least 125, at least 130 genes, at least 135 genes, at least 140 genes, at least 145, at least 150 genes, at least 155 genes, at least 160 genes, at least 165, at least 170 genes, at least 175 genes, or at least 180 genes, in a cancer; and producing a report, wherein the report identifies whether the cancer has a TITR gene signature using an over-representation/enrichment method; and wherein the genes are selected from CCR8, CD80, CSF2RB, ENTPD1, EBI3, TNFRSF8, TNFRSF18, DUSP4, SDC4, LAPTM4B, TNFRSF4, SEC14L1, TNFRSF9, IRAK2, FNDC3B, IKZF4, IL1R2, NINJ1, SYNGR2, TNIP2, SSH1, ARHGEF12, GCNT1, NCF4, MAPKAPK2, RHBDD2, TRAF3, IRF5, ACOT9, HIVEP3, RFK, UEVLD, BCL2L1, FAM129A, BATF, RHOC, ZNRF1, PMAIP1, PLAGL2, MRPS6, IL1RL1, CREB3L2, CPD, PICALM, CCR5, CTNNA1, GALC, TTC39C, CYFIP1, STAT1, FURIN, TRAF1, GBP5, HIF1A, UBE2L6, PTPRJ, TNFRSF1B, REM, CAPN2, TRIM16, CD74, MAP2K3, TIGIT, CTSH, TAP1, SKAP2, ITGAE, JAK3, CAPG, OPTN, NDRG1, DUSP16, CD274, ICOS, GLRX, SAMSN1, ANXA4, MAPKAPK3, KATNAL1, CDKN1A, SNX9. PFKL, ABI2, ATP2B4, GM2A, PGM2, RIPK3, ISGJ5, NAMPT, MVP, GOT1, MXD1, TMBIM1, RORA, PCYT1A, N4BP1, LAMP2, TMEM159, OSBPL3, VIM, ATF3, CXCR3, ALDOC, PLP2, FAM126A, OXSR1, GRN, APAF1, ANK3, ANKRD55, ANXA2R, ATM, BAZ2B, CCR7, CD200, CECR1, CLUHP3, CXCR5, DPH5, EPHA1, EPHA4, FAM174B, FAM65B, FASTKD2, FOXP1, GIMAP4, GIMAP5, GIMAP6, GIMAP7, GIMAP8, GZMK, HIVEP2, IL10, INADL, INPP4B, IQGAP2, KCNA3, KIAA1009, KIAA2026, KLF3, KLRG1, LDLRAP1, LEF1, LMO7, LOC100130231, LPAR6, LRIG1, LY9, MAP3K3, MS4A1, NAP1L2, OBSCN, PAR-SN, PLAC8, PLAG1, PLEK, PLK2, PLXDC1, PRKCQ-AS1, RNF214, SERINC5, SESN, SLAMF7, SLC16A7, SLC9A6, SMAD3, SPINT2, SSBP2, STXBP5, TCF7, TENM1, TMEM243, TRAT, TTC21B, TXK, ZBTB10, ZNF248, ZNF480, ZNF737, ZNF780B, and ZNF91.

### BRIEF DESCRIPTION OF THE DRAWINGS

FIG. 1 is a schematic illustrating the mechanism of action of a therapeutic combination described herein.

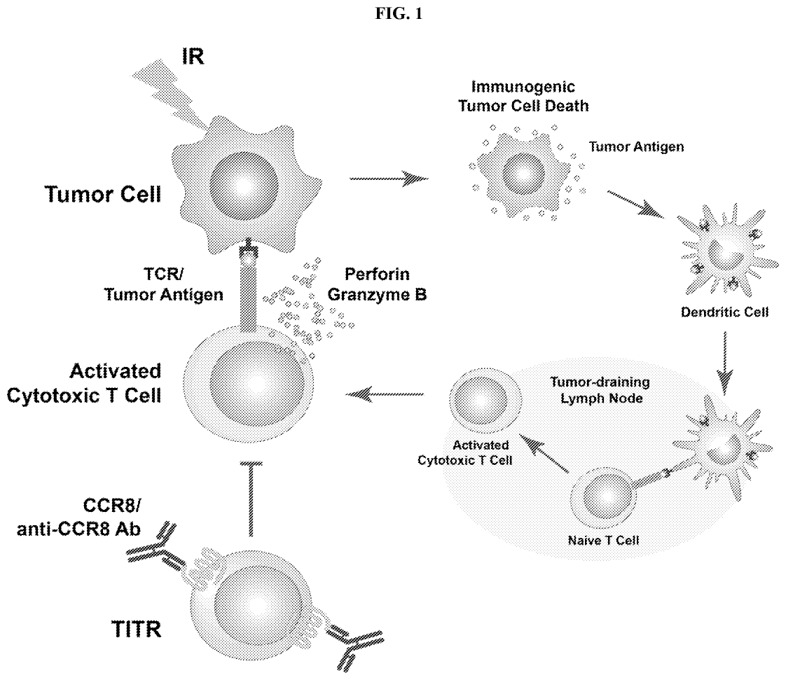

FIG. 2 shows percent viability of CD4⁺ conventional T cells, CD8⁺ T cells, and tumor infiltrating Tregs in ex vivo irradiated (0, 1, 2, and 4 Gy) human head and neck squamous cell carcinoma samples.

FIGS. 3A-3D show percent CCR8 positive cells in different populations of T cells in human tumor samples following ex vivo radiation treatment at 0, 2, and 4 Gy. CCR8 expression is highly enriched on TITR.

FIGS. 4A-4C show results of immunophenotyping by flow cytometry of excised and dissociated tumors from the syngeneic orthotopic breast cancer model. Combination of anti-CCR8 antibody+10 Gy RT lead to an increase in % CD8⁺ T Cells and decrease in % TITR cells over anti-CCR8 antibody treatment alone.

FIG. 5 shows results of immunophenotyping by flow cytometry of excised and dissociated tumors from the syngeneic orthotopic breast cancer model. Combination of anti-CCR8 antibody+10 Gy RT lead to increased activation level of CD8⁺ T cells by T cell activation marker CD39 over anti-CCR8 antibody treatment alone.

FIG. 6 shows results of an efficacy analysis of the combination of anti-CCR8 antibody with 10 Gy radiation therapy in vivo in a syngeneic orthotopic breast cancer model. A syngeneic orthotopic model of 4T1-Luc cells injected into the mammary fat pad was used as a breast cancer model to determine the efficacy of anti-CCR8 antibody therapy with radiation therapy.

FIG. 7 shows the result of a Kaplan-Meier survival analysis of mice following control (Ctrl) treatment, anti-CCR8 antibody alone (CCR8), 10 Gy radiation alone (Ctrl+10 Gy), and anti-CCR8 antibody+10 Gy (CCR8+10 Gy) combination treatment in vivo in a syngeneic orthotopic breast cancer model.

FIGS. 8A-8C show results of gene set enrichment analysis (GSEA) performed based on an RNA-sequencing study using dissociated tumor samples from the syngeneic orthotopic breast cancer model. Immune-associated gene sets are upregulated in anti-CCR8 antibody plus RT treated mouse tumor samples.

FIGS. 9A-9D show results of an RNA-sequencing study performed on dissociated tumor samples from the syngeneic orthotopic breast cancer model, indicating changes of TITR and CD8+ T cell gene signatures in breast tumors after anti-CCR8 antibody treatment in combination with radiation therapy. Shown are expression pattern of 108 TITR upregulated gene signature (FIG. 9A), 73 TITR downregulated gene signature (FIG. 9B), 180 total of TITR upregulated and downregulated gene signature (FIG. 9C) and CD8⁺ T cell gene signature (FIG. 9D) across control (Ctrl), anti-CCR8 antibody (CCR8), control+5 Gy, anti-CCR8 antibody+5 Gy, control+10 Gy, and anti-CCR8 antibody+10 Gy treatment groups.

FIG. 10 shows results of an efficacy analysis of the combination of anti-CCR8 antibody with 10 Gy focal radiation therapy in vivo in a lung cancer model. Murine Lewis lung carcinoma (LL/2) were subcutaneously implanted in the axilla (high) serving as a lung cancer model to determine the efficacy of anti-CCR8 antibody therapy with radiation therapy. Values are reported as means±SEM.

FIG. 11 shows the result of a Kaplan-Meier survival analysis of mice following control (Ctrl) treatment, anti-CCR8 antibody alone (CCR8), 10 Gy radiation therapy alone (Isotype ctrl+10 Gy), and anti-CCR8 antibody+10 Gy radiation therapy (CCR8+10 Gy) combination treatment in vivo in a lung cancer model.

FIG. 12 shows results of an efficacy analysis of the combination of anti-CCR8 antibody with anti-PD-1 antibody and 10 Gy focal radiation therapy in vivo in a lung cancer model. Murine Lewis lung carcinoma (LL/2) were subcutaneously implanted in the axilla (high) serving as a lung cancer model to determine the efficacy of anti-CCR8 antibody therapy with anti-PD-1 antibody and radiation therapy.

FIG. 13 shows the result of a Kaplan-Meier survival analysis of mice following control (Ctrl) treatment, anti-CCR8 antibody alone (CCR8), anti-CCR8 antibody+ anti-PD-1 antibody (CCR8+ PD-1), 10 Gy radiation therapy (Isotype ctrl+10 Gy), and anti-CCR8 antibody+anti-PD-1 antibody+10 Gy radiation therapy (CCR8+ PD-1+10 Gy) combination treatment in vivo in a lung cancer model. Values are reported as means±SEM.

FIG. 14 shows results of an efficacy analysis of the combination of (Ctrl) treatment, anti-CCR8 antibody alone (CCR8), 10 Gy radiation therapy alone (Isotype ctrl+10 Gy), and anti-CCR8 antibody+10 Gy radiation therapy (CCR8+10 Gy) in vivo in a breast cancer model. A syngeneic orthotopic model of 4T1-Luc cells injected into the mammary fat pad was used as a breast cancer model to determine the efficacy of anti-CCR8 antibody therapy with radiation therapy as measured by quantification of the number of tumor masses observed in various organs.

### DETAILED DESCRIPTION

Provided herein are therapeutic combinations of tumor infiltrating regulatory T cell (TITR) effectors with radiation therapy and methods of using the same for the treatment of cancer or for the targeting of TITR cells. Also provided herein are biomarkers for the selection of a patient to receive a therapeutic combination comprising a TITR effector and radiation therapy, as well as biomarkers for the assessment of the efficacy of a therapeutic combination comprising a TITR effector and radiation therapy.

### Therapeutic Combinations

In one aspect, provided herein is a method of targeting TITR cells in a subject. Without wishing to be bound by theory, it is believed that the preferential targeting of TITR cells offers several therapeutic advantages over the pan-Treg targeting. For example, it is hypothesized that TITR cells are more sensitive to radiation therapy, allowing for a lower dose of therapy to be administered. In some embodiments, the subject is afflicted with cancer, e.g., lung cancer, head and neck cancer, colon cancer brain cancer (e.g., glioblastoma), or skin cancer.

In another aspect, provided herein is a method of treating cancer in a subject, the method comprising administering to the subject a tumor infiltrating regulatory T cell (TITR) effector and radiation therapy. Also provided is the use of a TITR effector and radiation therapy for the treatment of cancer. Furthermore, provided herein is a combination comprising or consisting of a TITR effector and radiation therapy for use in the treatment of cancer. Further provided herein is the use of a TITR effector for use in the manufacture of a medicament for in combination with radiation therapy in the treatment of cancer.

As used herein, the term “TITR effector” means any compound or composition that decreases the population of TITRs in a tumor. In preferred embodiments, the TITR effector does not substantially decrease the population of regulatory T cells (Tregs). In some embodiments, the TITR effector is a CCR8 targeting agent, for example, an anti-CCR8 antibody. In some embodiments, the TITR effector is not a CCR8 targeting agent.

In some embodiments, a TITR effector decreases the population of TITRs in a cancer by about 5%, about 10%, about 20%, about 30%, about 40%, about 50%, about 60%, about 70%, about 80%, about 90%, about 5-10%, about 10-20%, about 20-30%, about 30-40%, about 40-50%, about 50-60%, about 60-70%, about 70-80%, about 80-90%, or about 90-100%. In some embodiments, no TITR cells are detectable in a cancer after administration of the TITR effector.

TITR cells may be detected using any suitable method known in the art or described herein. For example, TITR cells may be identified and qualified using RNAseq to detect a cell population with a TITR signature. Examples of genes that are upregulated or dowregulated in TITR cells are provided below.

Any suitable type of radiation therapy may be used in combination with a TITR effector. In some embodiments, the radiation therapy comprises the administration of electron radiation, proton radiation, or photon radiation, or any combination thereof. In some embodiments, the radiation therapy comprises administering ultra-high dose (FLASH) radiation therapy, e.g., electron FLASH, proton FLASH, or photon FLASH.

The radiation therapy of the methods described herein may be administered using any suitable system (or device) known in the art including, for example, an electron linear accelerator, a proton source, or an x-ray source. For example, radiation therapy may be delivered using electrons delivered by a linear electron accelerator is described, for example, in Favaudon et al., Transl. Med. 6, 245ra93 (2014). In other embodiments, the radiation therapy is administered using high energy charged particles, electrons, protons, heavy ions, high energy photons, x-rays, gamma rays, or neutrons.

Proton beam treatment has the advantage of being able to penetrate deeper into the tissue than electron beams. Furthermore, proton beams deposit the maximum of their energy at the end of their path, avoiding further penetration into healthy tissue (Liu, Chin J Cancer. 2011 May; 30(5): 315-326). Proton radiation therapy may be administered using a passive beam scattering system (e.g., a single scattering system or double scattering system) or a dynamic spot scanning system. In some embodiments, the radiation therapy or treatment system used to deliver proton radiation therapy is a proton pencil beam scanning system.

Exemplary devices that may be used to administer radiation therapy are described in, for example, U.S. Pat. No. 9,855,445, which is incorporated by reference herein in its entirety for the systems that may be used in the methods described herein. In some embodiments, the system used to administer radiation therapy in accordance with a method described herein comprises a nozzle, an accelerator, and a beam transport system. The nozzle may further comprise a scanning magnet, which guides the beam towards the target, and a beam energy adjuster. The accelerator may be based on radio frequency (e.g., a linear accelerator, a cyclotron, or a synchrotron) or a laser-based accelerator.

The dose of radiation therapy that is administered to a subject treated in accordance with a method described herein may depended on the characteristics of the subject and the cancer being treated. Without wishing to be bound by theory, it is believed that FLASH radiation therapy may be delivered at substantially higher doses than conventional dose rate radiation therapy due to its decreased normal tissue toxicity. As used herein, the term “conventional dose rate radiation therapy” is used to refer to radiation therapy that is administered at rates of about 2 Gy/see or less. One of skill in the art will appreciate that the radiation dose of conventional dose rate therapy that is administered will depend on many variables, including, without limitation, the tumor being treated, the stage and/or progression of the disease, patient co-morbidities, concurrent treatments, the device used to administer the radiation, and prior therapies. “FLASH” radiation therapy is generally administered at dose rates of at least 40 Gy/s.

In some embodiments, the dose rate of FLASH or conventional radiation therapy administered is at least 1 Gy/s. In some embodiments, the dose rate of FLASH or conventional radiation therapy administered is between 1 Gy/s and 60 Gy/s. In some embodiments, the dose rate of FLASH or conventional radiation therapy administered is about 1 Gy/s to about 5 Gy/s, about 5 Gy/s to about 10 Gy/s, about 10 Gy/s to about 15 Gy/s, about 15 Gy/s to about 20 Gy/s, about 20 Gy/s to about 25 Gy/s, about 25 Gy/s to about 30 Gy/s, about 30 Gy/s to about 35 Gy/s, about 35 Gy/s to about 40 Gy/s, about 40 Gy/s to about 45 Gy/s, about 45 Gy/s to about 50 Gy/s, about 50 Gy/s to about 55 Gy/s, about 55 Gy/s to about 60 Gy/s, about 60 Gy/s to about 65 Gy/s, or about 65 Gy/s to about 70 Gy/s.

In some embodiments, the dose of FLASH or conventional radiation therapy administered is between 1 Gy and 60 Gy. In some embodiments, the dose of FLASH or conventional radiation therapy administered is about 1 Gy to about 5 Gy, about 5 Gy to about 10 Gy, about 10 Gy to about 15 Gy, about 15 Gy to about 20 Gy, about 20 Gy to about 25 Gy, about 25 Gy to about 30 Gy, about 30 Gy to about 35 Gy, about 35 Gy to about 40 Gy, about 40 Gy to about 45 Gy, about 45 Gy to about 50 Gy, about 50 Gy to about 55 Gy, about 55 Gy to about 60 Gy, about 60 Gy to about 65 Gy, or about 65 Gy to about 70 Gy.

The radiation therapy may be delivered in a pulsed manner, a continuous manner, or a quasi-continuous manner. In some embodiments, the radiation therapy is administered in a pulsed manner with pulses at a frequency of about 100 Hz. In some embodiments, the dose of radiation therapy is delivered in a single pulse. In some embodiments, the dose of radiation therapy is delivered in a series of two or more pulses. Each pulse can have a duration of less than a second, several seconds, or several minutes. The interval between pulses may also last less than a second, several seconds, or several minutes. In some embodiments, each pulse in a series of pulses has the same duration. In some embodiments, the pulses in a series of pulses have different durations. In some embodiments, the intervals between each pulse in a series of pulses have the same duration. In some embodiments, the intervals between pulses in a series of pulses have different durations.

The dose and pulse parameters may be varied by a person skilled in the art to optimize the therapeutic effect. In some embodiments, the dose per pulse is at least 1 Gy, at least 2 Gy, at least 3 Gy, at least 4 Gy, or at least 5 Gy.

In some embodiments, the dose of radiation therapy is administered as fractionated doses, i.e., in a series of small doses over a period of time. Dose fractionation is used with conventional radiation therapy to reduce the incidence of radiation-induced side effects. Generally, a dose of conventional radiation therapy is fractionated into daily doses administered over weeks in order to achieve an acceptable therapeutic index (Dutt et al., Semin Radiat Oncol. 2020 Apr. 30(2): 194-200). Commonly, conventional therapy for solid tumors (e.g., standard of care radiation therapy) is fractionated into small doses of 1.8-2 Gy per day, delivered 5 days per week over the course of 6 to 8 weeks, resulting in a total doses of 60-80 Gy (Dutt et al., 2020)). Fractions of radiation may also be administered twice a day (at least 6 hours apart). Such “hyperfractionated” radiation therapy uses smaller fractions of, for example, about 1.5 Gy. In some embodiments, spatially fractionated radiation therapy (SFRT) is administered.

Without wishing to be bound by theory, it is believed that FLASH radiation therapy may be administered with fewer fractions than conventional dose rate radiation therapy due to its decreased healthy tissue toxicity. In some embodiments, no more than five fractions of FLASH radiation therapy are administered. In some embodiments, two fractions of FLASH radiation therapy are administered. In some embodiments, three fractions of FLASH radiation therapy are administered. In some embodiments, four fractions of FLASH radiation therapy are administered. In some embodiments, five fractions of FLASH radiation therapy are administered.

In some embodiments, the radiation therapy administered in combination with a TITR effector comprises the administration of one or more radionuclides. Examples of radionucleotides include sealed sources (e.g., brachytherapy) as well as unsealed sources (e.g., radiopharmaceuticals).

In some embodiment, additional therapeutic agents are administered in combination with the TITR effector and/or the radiation therapy. In some embodiments, the additional therapeutic agent is a checkpoint inhibitor. The term “inhibition” or “inhibitor” includes a reduction in a certain parameter, e.g., an activity, of a given molecule, e.g., an immune checkpoint inhibitor. For example, inhibition of an activity, e.g., an activity of, e.g., PD-1, PD-L1, CTLA-4, TIM-3, CEACAM (e.g., CEACAM-1, CEACAM-3 and/or CEACAM-5), LAG-3, VISTA, BTLA, TIGIT, LAIR1, CD160, 2B4 or TGFR beta, of at least 5%, 10%, 20%, 30%, 40%, 50% or more is included by this term. The level of inhibition need not be 100%.

In some embodiments, the checkpoint inhibitor is a PD-1 inhibitor. In some embodiments, the PD-1 inhibitor is an anti-PD1 antibody. In some embodiments, the PD-1 inhibitor is an anti PD-1 monoclonal antibody. Exemplary anti-PD-1 monoclonal antibodies include, but are not limited to cemiplimab (Libtayo), nivolumab (Opdivo), pembrolizumab (Keytruda).

The TITR effector and the radiation therapy may be administered concurrently or sequentially. In some embodiments, the TITR effector is administered before the radiation therapy. In some embodiments, the TITR effector is administered after the radiation therapy. The TITR effector, the radiation therapy, or both, may be administered repeatedly. In some embodiments, the TITR effector and/or the radiation therapy are administered at least one time, at least two times, at least 3 times, at least 4 times or at least 5 times. In some embodiments, the TITR effector and/or the radiation therapy are administered at least once every day, at least once every 2 days, at least once every 3 days, at least once every 4 days, at least once every 5 days, at least once every 6 days or at least once every 7 days. In some embodiments, the TITR effector and/or the radiation therapy are administered at least once every week, at least once every 2 weeks, at least once every 3 weeks, at least once every 4 weeks. If a TITR effector is an approved cancer therapeutic, the TITR effector may be administered according to the approved protocol.

The TITR effector, the radiation therapy and the checkpoint inhibitor may be administered concurrently or sequentially. In some embodiments, the TITR effector is administered before the radiation therapy. In some embodiments, the TITR effector is administered before the checkpoint inhibitor. In some embodiments, the TITR effector is administered after the radiation therapy. In some embodiments, the TITR effector is administered after the checkpoint inhibitor. In some embodiments the radiation therapy is administered before the checkpoint inhibitor. In some embodiments, the radiation therapy is administered after the checkpoint inhibit. The TITR effector, the radiation therapy and/or the checkpoint inhibitor may be administered repeatedly. In some embodiments, the TITR effector, the radiation therapy and/or the checkpoint inhibitor are administered at least one time, at least two times, at least 3 times, at least 4 times or at least 5 times. In some embodiments, the TITR effector, the radiation therapy and/or the checkpoint inhibitor are administered at least once every day, at least once every 2 days, at least once every 3 days, at least once every 4 days, at least once every 5 days, at least once every 6 days or at least once every 7 days. In some embodiments, the TITR effector, the radiation therapy and/or the checkpoint inhibitor are administered at least once every week, at least once every 2 weeks, at least once every 3 weeks, at least once every 4 weeks. If a TITR inhibitor is an approved cancer therapeutic, the TITR effector may be administered according to the approved protocol. If a checkpoint inhibitor is an approved cancer therapeutic, the checkpoint inhibitor may be administered according to the approved protocol.

In some embodiments, the therapeutic combination exhibits synergy, which allows for the dose of either the TITR effector or the radiation therapy or both to be administered at a lower dose than each would be administered as a single agent.

In some embodiments, the cancer treated in accordance with the methods described herein is lung cancer, head and neck cancer, colon cancer brain cancer (e.g., glioblastoma), or skin cancer.

In some embodiments, the cancer has previously been treated with a TITR effector and/or radiation therapy. In some embodiments, the cancer is resistant to a TITR effector and/or radiation therapy. Resistance to a TITR effector or radiation therapy may be intrinsic or acquired. In some embodiments, the cancer is resistant to the TITR effector which is used in the therapeutic combination. In some embodiments, the cancer is resistant to a different TITR effector to that which is used in the therapeutic combination. In some embodiments, the cancer is resistant to the type of radiation therapy which is used in the therapeutic combination. In some embodiments, the cancer is resistant to a different type of radiation therapy to that which is used in the therapeutic combination.

The terms “patient” and “subject” are used interchangeably herein. In preferred embodiments, the subject is human. In some embodiments, the subject is a human adult. In some embodiments, the subject is a human child. In some embodiments, the patient has undergone prior therapy for cancer, e.g., prior radiation therapy, prior chemotherapy, or a combination thereof. In some embodiments, the patient's cancer has recurred after the prior therapy (e.g., after prior radiation therapy, prior chemotherapy, or a combination thereof). In some embodiments, a patient's cancer has recurred after ablative radiation therapy. In some embodiments, the cancer is refractory to immune checkpoint blockade.

The efficacy of a method of treatment or a method of targeting TITR cells described herein may be evaluated using any suitable method known in the art or described herein. A method of treatment or a method of targeting TITR cells described herein may result the slowing or stopping of tumor growth, the decrease in tumor size, or any other suitable clinical endpoints that indicate therapeutic efficacy.

In some embodiments, a method of treatment or a method of targeting TITR cells described herein results in a decrease in tumor size of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% compared to the size of the tumor before the administration of the therapeutic combination. In some embodiments, a method of treatment or a method of targeting TITR cells described herein results in a decrease in tumor size of more than 95% compared to the size of the tumor before the administration of the therapeutic combination. In some embodiments, a method of treatment or a method of targeting TITR cells described herein results in a decrease in tumor size of about 5-10%, about 10-15%, about 15-20%, about 20-25%, about 25-30%, about 30-35%, about 35-40%, about 40-45%, about 45-50%, about 50-55%, about 55-60%, about 60-65%, about 65-70%, about 70-75%, about 75-80%, about 80-85%, about 85-90%, about 90-95%, or about 95-100% compared to the size of the tumor before the administration of the therapeutic combination. The tumor size may be determined at any suitable time point after treatment, for example, 1 month, 2 months, 3 months, 4 months, 5 months, or 6 months after the administration of the therapeutic combination.

In some embodiments, a method of treatment or a method of targeting TITR cells described herein result in delayed tumor recurrence. Generally, tumor recurrence refers to a tumor becoming detectable again after being undetectable for a prolonged period of time. In some embodiments, a tumor treated according to a method described herein does not recur for about 2 month to about 6 month, about 6 months to about 9 months, about 9 months to about 12 months, about 12 months to about 15 months, about 15 months to about 18 months, about 18 months to about 21 months, about 21 months to about 24 months, about 2 years to about 3 years, about 3 years to about 4 years, about 4 years to about 5 years, or about 5 years to about 10 years. In some embodiments, a tumor treated according to a method described herein does not recur for at least about 2 month, at least about 3 month, at least about 6 month, at least about 9 months, at least about 12 months, at least about 15 months, at least about 18 months, at least about 21 months, at least about 2 years, at least about 3 years, at least about 4 years, at least about 5 years, or at least about 10 years.

In some embodiments, a method of treatment or a method of targeting TITR cells described herein results in a decrease in number and/or size of tumor metastases of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% compared to the number and/or size of tumor metastases before the administration of the therapeutic combination. In some embodiments, a method of treatment or a method of targeting TITR cells described herein results in a decrease in number and/or size of tumor metastases of about 5-10%, about 10-15%, about 15-20%, about 20-25%, about 25-30%, about 30-35%, about 35-40%, about 40-45%, about 45-50%, about 50-55%, about 55-60%, about 60-65%, about 65-70%, about 70-75%, about 75-80%, about 80-85%, about 85-90%, about 90-95%, or about 95-100% compared to the number and/or size of tumor metastases before the administration of the therapeutic combination. The number and/or size tumor metastasis may be determined at any suitable time point after treatment, for example, 1 month, 2 months, 3 months, 4 months, 5 months, or 6 months after the administration of the therapeutic combination. In some embodiments, a method of treatment described herein prevents the occurrence of metastases for a least 6 months, at least 9 months, at least 12 months, at least 2 years, or at least 3 years.

Tumors may be detected and measured using computer tomography (CT) scanning, magnetic resonance imagining (MRI), positron emission tomography (PET), x-ray, or physical examination, or any other suitable method known in the art.

In some embodiments, a therapeutic combination described herein induces immunogenic cell death in the cancer.

### Biomarkers

In another aspect, provided herein are methods for selecting cancer patients for treatment and methods for determining if a cancer patient is responding to treatment. These methods rely on the identification and measurement of a TITR gene signature in a tumor and quantifying it prior to and after treatment. If a tumor shows increased expression of genes that upregulated in TITR, and/or decreased expression of genes downregulated in TITR, the tumor has a TITR gene signature. Similar methods can be used to determine whether a tumor has a Treg signature: If a tumor shows increased expression of genes that are highly expressed in Tregs, the tumor shows a Treg gene signature. If a tumor shows increased expression of genes that are highly expressed in CD8+ cells, the tumor is said to have a CD8 gene signature.

Increased gene expression of TITR-upregulated genes and/or downregulation of TITR-downregulated genes may be identified in the tumor compared to normal tissue using any suitable method known in the art, including, for example, an over-representation/enrichment method. Such a method determines whether genes of a particular set (e.g., TITR-downregulated or TITR-upregulated genes) are expressed more highly than would be expected, e.g., expected by chance. In some embodiments, the increased or decreased gene expression is determined using an over-representation method or an enrichment method (such as GSVA score or Xcell score).

In some embodiments, expression is determined in comparison to the expression levels of all genes in a sample that was not treated with radiation therapy or immuno-therapy.

Methods of determining gene expression are well known in the art. Any suitable method may be used to determine the expression of genes for the methods described herein, including, for example, RNA sequencing.

Genes that are upregulated in TITR include CCR8, CD80, CSF2RB, ENTPD1, EBI3, TNFRSF8, TNFRSF18, DUSP4, SDC4, LAPTM4B, TNFRSF4, SEC14L1, TNFRSF9, IRAK2, FNDC3B, IKZF4, IL1R2, NINJ1, SYNGR2, TNIP2, SSH1, ARHGEF12, GCNT1, NCF4, MAPKAPK2, RHBDD2, TRAF3, IRF5, ACOT9, HIVEP3, RFK, UEVLD, BCL2L1, FAM129A, BATF, RHOC, ZNRF1, PMAIP1, PLAGL2, MRPS6, IL1RL1, CREB3L2, CPD, PICALM, CCR5, CTNNA1, GALC, TTC39C, CYFIP1, STAT1, FURIN, TRAF1, GBP5, HIF1A, UBE2L6, PTPRJ, TNFRSF1B, REM, CAPN2, TRIM16, CD74, MAP2K3, TIGIT, CTSH, TAP1, SKAP2, ITGAE, JAK3, CAPG, OPTN, NDRG1, DUSP16, CD274, ICOS, GLRX, SAMSN1, ANXA4, MAPKAPK3, KATNAL1, CDKN1A, SNX9. PFKL, ABI2, ATP2B4, GM2A, PGM2, RIPK3, ISGJ5, NAMPT, MVP, GOT1, MXD1, TMBIM1, RORA, PCYT1A, N4BP1, LAMP2, TMEM159, OSBPL3, VIM, ATF3, CXCR3, ALDOC, PLP2, FAM126A, OXSR1, GRN, and APAF1. See Maguson et al., Proc Natl Acad Sci USA. 2018; 115(45): E10672-E10681.

Genes that are downregulated in TITR include ANK3, ANKRD55, ANXA2R, ATM, BAZ2B, CCR7, CD200, CECR1, CLUHP3, CXCR5, DPH5, EPHA1, EPHA4, FAM174B, FAM65B, FASTKD2, FOXP1, GIMAP4, GIMAP5, GIMAP6, GIMAP7, GIMAP8, GZMK, HIVEP2, IL10, INADL, INPP4B, IQGAP2, KCNA3, KIAA1009, KIAA2026, KLF3, KLRG1, LDLRAP1, LEF1, LMO7, LOC100130231, LPAR6, LRIG1, LY9, MAP3K3, MS4A1, NAP1L2, OBSCN, PAR-SN, PLAC8, PLAG1, PLEK, PLK2, PLXDC1, PRKCQ-AS1, RNF214, SERINC5, SESN1, SLAMF7, SLC16A7, SLC9A6, SMAD3, SPINT2, SSBP2, STXBP5, TCF7, TENM1, TMEM243, TRAT1, TTC21B, TXK, ZBTB10, ZNF248, ZNF480, ZNF737, ZNF780B, and ZNF91. See Maguson et al., Proc Natl Acad Sci USA. 2018; 115(45): E10672-E10681.

Genes that are highly expressed in Tregs include CCL19, CD34, CD72, CTLA4, FOXP3, GADD45B, GEM, IL1RL1, IL9R, MADCAM1, MYH10, NCF2, RCSD1, RYR1, SELE, SELP, SFRP1, SIT1, TIGIT, TLR10, TLR2, TLR7, TLR8, TRAF1, WIPF1, and TGFB1. See Angelova et al. Genome Biol. 2015; 16:64.

Genes that are highly expressed in CD8+ cells include CD8B, CD8A, CD8B, PF4, PRR5, SF1, LIME1, DNAJB1, ARHGAP8, GZMM, SLC16A7, SFRS7, APBA2, C4orf15, LEPROTL1, ZFP36L2, GADD45A, ZFP36L2, MYST3, ZEB1, ZNF609, C12orf47, THUMPD1, VAMP2, ZNF91, ZNF22, TMC6, DNAJB1, FLT3LG, CDKN2AIP, TSC22D3, TBCC, RBM3, ABT1, C19orf6, CAMLG, PPP1R2, AES, KLF9 and PRF1. See Bindea et al. Immunity. 2013; 39:782-95.

In one aspect, provided herein is a method of treating cancer in a subject, the method comprising (a) obtaining a sample from the cancer and a normal tissue sample from the subject; (b) quantifying the expression level of at least 3, at least 4, at least 5, at least 6, at least 7, at least 8, at least 9, at least 10, at least 11, at least 12, at least 13, at least 14, at least 15, at least 16, at least 17, at least 18, at least 19, at least 20 genes, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110 genes, at least 115 genes, at least 120 genes, at least 125, at least 130 genes, at least 135 genes, at least 140 genes, at least 145, at least 150 genes, at least 155 genes, at least 160 genes, at least 165, at least 170 genes, at least 175 genes, or at least 180 genes, wherein the genes are selected from: CCR8, CD80, CSF2RB, ENTPD1, EBI3, TNFRSF8, TNFRSF18, DUSP4, SDC4, LAPTM4B, TNFRSF4, SEC14L1, TNFRSF9, IRAK2, FNDC3B, IKZF4, IL1R2, NINJ1, SYNGR2, TNIP2, SSH1, ARHGEF12, GCNT1, NCF4, MAPKAPK2, RHBDD2, TRAF3, IRF5, ACOT9, HIVEP3, RFK, UEVLD, BCL2L1, FAM129A, BATF, RHOC, ZNRF1, PMAIP1, PLAGL2, MRPS6, IL1RL1, CREB3L2, CPD, PICALM, CCR5, CTNNA1, GALC, TTC39C, CYFIP1, STAT1, FURIN, TRAF1, GBP5, HIF1A, UBE2L6, PTPRJ, TNFRSF1B, REM, CAPN2, TRIM16, CD74, MAP2K3, TIGIT, CTSH, TAP1, SKAP2, ITGAE, JAK3, CAPG, OPTN, NDRG1, DUSP16, CD274, ICOS, GLRX, SAMSN1, ANXA4, MAPKAPK3, KATNAL1, CDKN1A, SNX9. PFKL, ABI2, ATP2B4, GM2A, PGM2, RIPK3, ISG15, NAMPT, MVP, GOT1, MXD1, TMBIM1, RORA, PCYT1A, N4BP1, LAMP2, TMEM159, OSBPL3, VIM, ATF3, CXCR3, ALDOC, PLP2, FAM126A, OXSR1, GRN, APAF1, ANK3, ANKRD55, ANXA2R, ATM, BAZ2B, CCR7, CD200, CECR1, CLUHP3, CXCR5, DPH5, EPHA1, EPHA4, FAM174B, FAM65B, FASTKD2, FOXP1, GIMAP4, GIMAP5, GIMAP6, GIMAP7, GIMAP8, GZMK, HIVEP2, IL10, INADL, INPP4B, IQGAP2, KCNA3, KIAA1009, KIAA2026, KLF3, KLRG1, LDLRAP1, LEF1, LMO7, LOC100130231, LPAR6, LRIG1, LY9, MAP3K3, MS4A1, NAP1L2, OBSCN, PAR-SN, PLAC8, PLAG1, PLEK, PLK2, PLXDC1, PRKCQ-AS1, RNF214, SERINC5, SESN1, SLAMF7, SLC16A7, SLC9A6, SMAD3, SPINT2, SSBP2, STXBP5, TCF7, TENM1, TMEM243, TRAT1, TTC21B, TXK, ZBTB10, ZNF248, ZNF480, ZNF737, ZNF780B, and ZNF91; and (c) administering to the subject a combination therapy comprising a TITR effector and radiation therapy if a TITR signature is identified in the tumor compared to normal tissue using an over-representation/enrichment method.

In another aspect, provided herein is a method of identifying a patient to be treated with a combination therapy comprising a TITR effector and radiation therapy, the method comprising (a) obtaining a sample from the cancer and a normal tissue sample; and (b) quantifying the expression level of at least 3, at least 4, at least 5, at least 6, at least 7, at least 8, at least 9, at least 10, at least 11, at least 12, at least 13, at least 14, at least 15, at least 16, at least 17, at least 18, at least 19, at least 20 genes, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110 genes, at least 115 genes, at least 120 genes, at least 125, at least 130 genes, at least 135 genes, at least 140 genes, at least 145, at least 150 genes, at least 155 genes, at least 160 genes, at least 165, at least 170 genes, at least 175 genes, or at least 180 genes wherein the genes are selected from: CCR8, CD80, CSF2RB, ENTPD1, EBI3, TNFRSF8, TNFRSF18, DUSP4, SDC4, LAPTM4B, TNFRSF4, SEC14L1, TNFRSF9, IRAK2, FNDC3B, IKZF4, IL1R2, NINJ1, SYNGR2, TNIP2, SSH1, ARHGEF12, GCNT1, NCF4, MAPKAPK2, RHBDD2, TRAF3, IRF5, ACOT9, HIVEP3, RFK, UEVLD, BCL2L1, FAM129A, BATF, RHOC, ZNRF1, PMAIP1, PLAGL2, MRPS6, IL1RL1, CREB3L2, CPD, PICALM, CCR5, CTNNA1, GALC, TTC39C, CYFIP1, STAT1, FURIN, TRAF1, GBP5, HIF1A, UBE2L6, PTPRJ, TNFRSF1B, REM, CAPN2, TRIM16, CD74, MAP2K3, TIGIT, CTSH, TAP1, SKAP2, ITGAE, JAK3, CAPG, OPTN, NDRG1, DUSP16, CD274, ICOS, GLRX, SAMSN1, ANXA4, MAPKAPK3, KATNAL1, CDKN1A, SNX9. PFKL, ABI2, ATP2B4, GM2A, PGM2, RIPK3, ISG15, NAMPT, MVP, GOT1, MXD1, TMBIM1, RORA, PCYT1A, N4BP1, LAMP2, TMEM159, OSBPL3, VIM, ATF3, CXCR3, ALDOC, PLP2, FAM126A, OXSR1, GRN, APAF1, ANK3, ANKRD55, ANXA2R, ATM, BAZ2B, CCR7, CD200, CECR1, CLUHP3, CXCR5, DPH5, EPHA1, EPHA4, FAM174B, FAM65B, FASTKD2, FOXP1, GIMAP4, GIMAP5, GIMAP6, GIMAP7, GIMAP8, GZMK, HIVEP2, IL10, INADL, INPP4B, IQGAP2, KCNA3, KIAA1009, KIAA2026, KLF3, KLRG1, LDLRAP1, LEF1, LMO7, LOC100130231, LPAR6, LRIG1, LY9, MAP3K3, MS4A1, NAP1L2, OBSCN, PAR-SN, PLAC8, PLAG1, PLEK, PLK2, PLXDC1, PRKCQ-AS1, RNF214, SERINC5, SESN1, SLAMF7, SLC16A7, SLC9A6, SMAD3, SPINT2, SSBP2, STXBP5, TCF7, TENM1, TMEM243, TRAT1, TTC21B, TXK, ZBTB10, ZNF248, ZNF480, ZNF737, ZNF780B, and ZNF91;

Increased gene expression of TITR-upregulated genes or downregulation of TITR-downregulated genes may indicate a TITR signature, and can be identified in the tumor compared to normal tissue using an over-representation/enrichment method.

In another aspect, provided herein is a method for selecting a patient for treatment with a combination therapy comprising a TITR effector and radiation therapy, the method comprising (a) obtaining a sample from the cancer and a normal tissue sample; (b) quantifying the expression level of at least 3, at least 4, at least 5, at least 6, at least 7, at least 8, at least 9, at least 10, at least 11, at least 12, at least 13, at least 14, at least 15, at least 16, at least 17, at least 18, at least 19, at least 20 genes, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110 genes, at least 115 genes, at least 120 genes, at least 125, at least 130 genes, at least 135 genes, at least 140 genes, at least 145, at least 150 genes, at least 155 genes, at least 160 genes, at least 165, at least 170 genes, at least 175 genes, or at least 180 genes wherein the genes are selected from: CCR8, CD80, CSF2RB, ENTPD1, EBI3, TNFRSF8, TNFRSF18, DUSP4, SDC4, LAPTM4B, TNFRSF4, SEC14L1, TNFRSF9, IRAK2, FNDC3B, IKZF4, IL1R2, NINJ1, SYNGR2, TNIP2, SSH1, ARHGEF12, GCNT1, NCF4, MAPKAPK2, RHBDD2, TRAF3, IRF5, ACOT9, HIVEP3, RFK, UEVLD, BCL2L1, FAM129A, BATF, RHOC, ZNRF1, PMAIP1, PLAGL2, MRPS6, IL1RL1, CREB3L2, CPD, PICALM, CCR5, CTNNA1, GALC, TTC39C, CYFIP1, STAT1, FURIN, TRAF1, GBP5, HIF1A, UBE2L6, PTPRJ, TNFRSF1B, REM, CAPN2, TRIM16, CD74, MAP2K3, TIGIT, CTSH, TAP1, SKAP2, ITGAE, JAK3, CAPG, OPTN, NDRG1, DUSP16, CD274, ICOS, GLRX, SAMSN1, ANXA4, MAPKAPK3, KATNAL1, CDKN1A, SNX9. PFKL, ABI2, ATP2B4, GM2A, PGM2, RIPK3, ISG15, NAMPT, MVP, GOT1, MXD1, TMBIM1, RORA, PCYT1A, N4BP1, LAMP2, TMEM159, OSBPL3, VIM, ATF3, CXCR3, ALDOC, PLP2, FAM126A, OXSR1, GRN, APAF1, ANK3, ANKRD55, ANXA2R, ATM, BAZ2B, CCR7, CD200, CECR1, CLUHP3, CXCR5, DPH5, EPHA1, EPHA4, FAM174B, FAM65B, FASTKD2, FOXP1, GIMAP4, GIMAP5, GIMAP6, GIMAP7, GIMAP8, GZMK, HIVEP2, IL10, INADL, INPP4B, IQGAP2, KCNA3, KIAA1009, KIAA2026, KLF3, KLRG1, LDLRAP1, LEF1, LMO7, LOC100130231, LPAR6, LRIG1, LY9, MAP3K3, MS4A1, NAP1L2, OBSCN, PAR-SN, PLAC8, PLAG1, PLEK, PLK2, PLXDC1, PRKCQ-AS1, RNF214, SERINC5, SESN1, SLAMF7, SLC16A7, SLC9A6, SMAD3, SPINT2, SSBP2, STXBP5, TCF7, TENM1, TMEM243, TRAT1, TTC21B, TXK, ZBTB10, ZNF248, ZNF480, ZNF737, ZNF780B, and ZNF91; and (c) selecting the patient for if a TITR gene signature is identified in the tumor compared to normal tissue using an over-representation/enrichment method.

Further provided is the use of a plurality of agents specific to quantify the expression level of at least 3, at least 4, at least 5, at least 6, at least 7, at least 8, at least 9, at least 10, at least 11, at least 12, at least 13, at least 14, at least 15, at least 16, at least 17, at least 18, at least 19, at least 20 genes, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110 genes, at least 115 genes, at least 120 genes, at least 125, at least 130 genes, at least 135 genes, at least 140 genes, at least 145, at least 150 genes, at least 155 genes, at least 160 genes, at least 165, at least 170 genes, at least 175 genes, or at least 180 genes wherein the genes are selected from: CCR8, CD80, CSF2RB, ENTPD1, EBI3, TNFRSF8, TNFRSF18, DUSP4, SDC4, LAPTM4B, TNFRSF4, SEC14L1, TNFRSF9, IRAK2, FNDC3B, IKZF4, IL1R2, NINJ1, SYNGR2, TNIP2, SSH1, ARHGEF2, GCNT1, NCF4, MAPKAPK2, RHBDD2, TRAF3, IRF5, ACOT9, HIVEP3, RFK, UEVLD, BCL2L1, FAM129A, BATF, RHOC, ZNRF1, PMAIP1, PLAGL2, MRPS6, IL1RL1, CREB3L2, CPD, PICALM, CCR5, CTNNA1, GALC, TTC39C, CYFIP1, STAT1, FURIN, TRAF1, GBP5, HIF1A, UBE2L6, PTPRJ, TNFRSF1B, REM, CAPN2, TRIM16, CD74, MAP2K3, TIGIT, CTSH, TAP1, SKAP2, ITGAE, JAK3, CAPG, OPTN, NDRG1, DUSP16, CD274, ICOS, GLRX, SAMSN1, ANXA4, MAPKAPK3, KATNAL1, CDKN1A, SNX9. PFKL, ABI2, ATP2B4, GM2A, PGM2, RIPK3, ISG15, NAMPT, MVP, GOT1, MXD1, TMBIM1, RORA, PCYT1A, N4BP1, LAMP2, TMEM159, OSBPL3, VIM, ATF3, CXCR3, ALDOC, PLP2, FAM126A, OXSR1, GRN, APAF1, ANK3, ANKRD55, ANXA2R, ATM, BAZ2B, CCR7, CD200, CECR1, CLUHP3, CXCR5, DPH5, EPHA1, EPHA4, FAM174B, FAM65B, FASTKD2, FOXP1, GIMAP4, GIMAP5, GIMAP6, GIMAP7, GIMAP8, GZMK, HIVEP2, IL10, INADL, INPP4B, IQGAP2, KCNA3, KIAA1009, KIAA2026, KLF3, KLRG1, LDLRAP1, LEF1, LMO7, LOC100130231, LPAR6, LRIG1, LY9, MAP3K3, MS4A1, NAP1L2, OBSCN, PAR-SN, PLAC8, PLAG1, PLEK, PLK2, PLXDC1, PRKCQ-AS1, RNF214, SERINC5, SESN1, SLAMF7, SLC16A7, SLC9A6, SMAD3, SPINT2, SSBP2, STXBP5, TCF7, TENM1, TMEM243, TRAT1, TTC21B, TXK, ZBTB10, ZNF248, ZNF480, ZNF737, ZNF780B, and ZNF91 in the manufacture of a kit for selecting a subject for treatment with a combination therapy comprising a TITR inhibitor and radiotherapy by a method comprising (a) obtaining a sample from the cancer and a normal tissue sample; (b) quantifying the expression level of the at least 3, at least 4, at least 5, at least 6, at least 7, at least 8, at least 9, at least 10, at least 11, at least 12, at least 13, at least 14, at least 15, at least 16, at least 17, at least 18, at least 19, at least 20 genes, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110 genes, at least 115 genes, at least 120 genes, at least 125, at least 130 genes, at least 135 genes, at least 140 genes, at least 145, at least 150 genes, at least 155 genes, at least 160 genes, at least 165, at least 170 genes, at least 175 genes, or at least 180 genes; and (c) selecting the patient for treatment if a TITR gene signature is identified in the tumor compared to normal tissue using an over-representation/enrichment method.

Further provided is the use of a plurality of agents specific to quantifying the expression level of at least 3, at least 4, at least 5, at least 6, at least 7, at least 8, at least 9, at least 10, at least 11, at least 12, at least 13, at least 14, at least 15, at least 16, at least 17, at least 18, at least 19, at least 20 genes, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110 genes, at least 115 genes, at least 120 genes, at least 125, at least 130 genes, at least 135 genes, at least 140 genes, at least 145, at least 150 genes, at least 155 genes, at least 160 genes, at least 165, at least 170 genes, at least 175 genes, or at least 180 genes wherein the genes are selected from: CCR8, CD80, CSF2RB, ENTPD1, EBI3, TNFRSF8, TNFRSF18, DUSP4, SDC4, LAPTM4B, TNFRSF4, SEC14L1, TNFRSF9, IRAK2, FNDC3B, IKZF4, IL1R2, NINJ1, SYNGR2, TNIP2, SSH1, ARHGEF12, GCNT1, NCF4, MAPKAPK2, RHBDD2, TRAF3, IRF5, ACOT9, HIVEP3, RFK, UEVLD, BCL2L1, FAM129A, BATF, RHOC, ZNRF1, PMAIP1, PLAGL2, MRPS6, IL1RL1, CREB3L2, CPD, PICALM, CCR5, CTNNA1, GALC, TTC39C, CYFIP1, STAT1, FURIN, TRAF1, GBP5, HIF1A, UBE2L6, PTPRJ, TNFRSF1B, REM, CAPN2, TRIM16, CD74, MAP2K3, TIGIT, CTSH, TAP1, SKAP2, ITGAE, JAK3, CAPG, OPTN, NDRG1, DUSP16, CD274, ICOS, GLRX, SAMSN1, ANXA4, MAPKAPK3, KATNAL1, CDKN1A, SNX9. PFKL, ABI2, ATP2B4, GM2A, PGM2, RIPK3, ISG15, NAMPT, MVP, GOT1, MXD1, TMBIM1, RORA, PCYT1A, N4BP1, LAMP2, TMEM159, OSBPL3, VIM, ATF3, CXCR3, ALDOC, PLP2, FAM126A, OXSR1, GRN, APAF1, ANK3, ANKRD55, ANXA2R, ATM, BAZ2B, CCR7, CD200, CECR1, CLUHP3, CXCR5, DPH5, EPHA1, EPHA4, FAM174B, FAM65B, FASTKD2, FOXP1, GIMAP4, GIMAP5, GIMAP6, GIMAP7, GIMAP8, GZMK, HIVEP2, IL10, INADL, INPP4B, IQGAP2, KCNA3, KIAA1009, KIAA2026, KLF3, KLRG1, LDLRAP1, LEF1, LMO7, LOC100130231, LPAR6, LRIG1, LY9, MAP3K3, MS4A1, NAP1L2, OBSCN, PAR-SN, PLAC8, PLAG1, PLEK, PLK2, PLXDC1, PRKCQ-AS1, RNF214, SERINC5, SESN1, SLAMF7, SLC16A7, SLC9A6, SMAD3, SPINT2, SSBP2, STXBP5, TCF7, TENM1, TMEM243, TRAT1, TTC21B, TXK, ZBTB10, ZNF248, ZNF480, ZNF737, ZNF780B, and ZNF91 in the manufacture of a kit for detecting and/or quantifying a TITR gene signature in a cancer by a method comprising (a) obtaining a sample from the cancer and a normal tissue sample; (b) quantifying the expression level of the at least 3, at least 4, at least 5, at least 6, at least 7, at least 8, at least 9, at least 10, at least 11, at least 12, at least 13, at least 14, at least 15, at least 16, at least 17, at least 18, at least 19, at least 20 genes, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110 genes, at least 115 genes, at least 120 genes, at least 125, at least 130 genes, at least 135 genes, at least 140 genes, at least 145, at least 150 genes, at least 155 genes, at least 160 genes, at least 165, at least 170 genes, at least 175 genes, or at least 180 genes; and (c) producing a report, wherein the report identifies whether the cancer has a TITR gene signature compared to normal tissue using an over-representation/enrichment method.

The gene signatures provided herein may also be used to determine whether a patient is responding to a therapeutic combination comprising a TITR effector and radiation therapy. In some embodiments, treatment in accordance with a method described herein results in a decrease in the expression of genes upregulated in TITR in the cancer. In some embodiments, treatment in accordance with a method described herein results in an increase in the expression of genes downregulated in TITR. In some embodiments, treatment in accordance with a method described herein results in an increase in the expression of genes that are highly expressed in Treg cells.

The methods provided herein may require the comparison of gene expression in a sample from the cancer and a normal tissue sample. The samples may be obtained by any suitable method. In some embodiments, the sample from the cancer is obtained by biopsy, for example, needle biopsy, open biopsy, punch biopsy, lymph node biopsy or bone marrow aspiration. In some embodiments, the normal tissue sample is taken from the same subject as the cancer sample. In some embodiments, the normal tissue sample is taken from an unaffected part of the same organ as the cancer, for example, if the subject is afflicted with lung cancer, the normal tissue sample is taken from an unaffected part of the lung.

### EXAMPLES

The examples in this section are provided for illustration only and are not intended to limit the invention in any way.

### Example 1: Combination of TITR Effectors with Radiation Therapy to Treat Cancer

The aim of this study was to evaluate the efficacy of targeting TITR cells in combination with radiation therapy in a mouse syngeneic orthotopic model of breast cancer.

### Materials and Methods

### Human Tumor Procurement

Human tumor samples were provided by the Cooperative Human Tissue Network (CHTN) which is funded by the National Cancer Institute. Tumor samples were shipped overnight in RPMI+10% FBS on cold packs.

### Human Tumor Histoculture

Fresh human head and neck squamous cell carcinoma samples were embedded in 0.02 mg/mL agarose solution prior to slicing at 300-400 μm thickness using a vibrating microtome (Precisionary Instruments #VF-310-0Z). The sliced human tumor samples were then cultured on a piece of Avitene Ultrafoam collagen sponge (Becton Dickinson) in one well of a 6-well cell culture plate containing 5 mL of culture media (RPMI+10% FBS+1 mM Sodium Pyruvate+1× GlutaMAX+1× Antibiotic-antimycotic). Human tumor slices were irradiated ex vivo using a cabinet X-ray irradiator (Precision X-Ray) followed by incubation on a plate shaker at 37° C.

### In Vivo Study Protocol—Orthotopic Breast Cancer Model

Female Balb/c mice, 6-7 weeks old, were orthotopically implanted with 500000 4T1-Luc2-1A4 cells in mammary fat pad #4. Animals were injected intraperitoneally with 10 mg/kg anti-CCR8 (BioLegend 96199, custom order of clone SA214G2) or Isotype control antibody (BioLegend 400668) on Days 7, 10, and 14 post-implantation. Irradiated groups received 5 Gy or 10 Gy focal radiation delivered by Xstrahl SARRP on Days 7, 10, and 14 post-implantation. Tumors, draining lymph nodes, and blood were harvested for flow cytometric analysis on Day 15 post-implantation. Tumor measurements were taken 3 times per week for efficacy evaluation.

### In Vivo Study Protocol Subcutaneous Lung Cancer Model

Female C57BL/6 (C57BL/6NHsd) mice, 7-8 weeks old, were subcutaneously implanted with 1,000,000 Murine Lewis lung carcinoma (LL/2) in the axilla (high). Animals were injected intraperitoneally with 10 mg/kg anti-CCR8 (BioLegend 150302, custom order of clone SA214G2), and/or 10 mg/Kg anti-PD-1 (BioXCell BP0146, Clone RMP1-14) or 10 mg/kg Isotype control abitody (BioXell BP0089, clone 2A3) on days 8, 11, 14 and 17 post-implantation. Irradiated groups received 10 Gy focal radiation delivered by Xstrahl SARRP on Day 8 post-implantation. Tumor measurements were taken 3 times per week with calipers for efficacy evaluation.

### Biospecimen Dissociation:

Tumors and draining lymph nodes were mechanically and enzymatically dissociated with Miltenyi Mouse Tumor Dissociation mix according to manufacturer's protocol, reducing R component to 20%. Human tumor histoculture samples were enzymatically dissociated using components from Miltenyi Human Tumor Dissociation Kit on the gentleMACS Dissociator (Miltenyi Biotec).

### Flow Cytometric Analysis

Flow cytometric studies to analyze human or mouse tumor infiltrating lymphocytes were performed on dissociated human or mouse tumor cells. Dissociated human tumor cells were stained with a panel of twenty-one fluorescent-labeled anti-human antibodies listed in Table 1. Dissociated cells from mouse tumor samples, lymph node samples, and peripheral blood samples were stained with a panel of twenty-one fluorescent-labeled anti-mouse antibodies listed in Table 2. Post staining, cells were resuspended in PBS+1% FBS+2 mM EDTA and analyzed on a BD FACSymphony™ A3 Cell Analyzer.

TABLE 1
List of Human Antibodies

| ANTIBODIES | SOURCE | IDENTIFIER |
|---|---|---|
| Alexa Fluor ®647 Mouse Anti-Human | BD Biosciences | Cat. 557711 |
| CD56 | | RRID: AB_396820 |
| APC-R700 Mouse Anti-Human CD25 | BD Biosciences | Cat. 565106 |
| | | RRID: AB_2744339 |
| APC-H7 Mouse Anti-Human HLA-DR | BD Biosciences | Cat. 561358 |
| | | RRID: AB_10611876 |
| AF488 Mouse Anti-Human Granzyme B | Invitrogen | Cat. MA5-23639 |
| | | RRID: AB_2609543 |
| BB700 Mouse Anti-Human CD141 | BD Biosciences | Cat. 742245 |
| | | RRID: AB_2740668 |
| BUV395 Mouse Anti-Human CD45 | BD Biosciences | Cat. 563792 |
| | | RRID: AB_2869519 |
| BUV496 Mouse Anti-Human CD4 | BD Biosciences | Cat. 612936 |
| | | RRID: AB_2870220 |
| BUV563 Mouse Anti-Human HLA-ABC | BD Biosciences | Cat. 741378 |
| | | RRID: AB_2870877 |
| BUV661 Mouse Anti-Human CD15 | BD Biosciences | Cat. 741660 |
| | | RRID: AB_2871054 |
| BUV737 Mouse Anti-Human CD86 | BD Biosciences | Cat. 612784 |
| | | RRID: AB_2814790 |
| BUV805 Mouse Anti-Human CD3 | BD Biosciences | Cat. 612895 |
| | | RRID: AB_2870183 |
| BV421 Mouse Anti-Ki-67 | BD Biosciences | Cat. 562899 |
| | | RRID: AB_2686897 |
| BV480 Mouse Anti-Human CD8 | BD Biosciences | Cat. 566121 |
| | | RRID: AB_2739523 |
| Fixable Viability Stain 575V | BD Biosciences | Cat. 565694 |
| | | RRID: AB_2869702 |
| BV650 Mouse Anti-Human CD11c | BD Biosciences | Cat. 563404 |
| | | RRID: AB_2869490 |
| BV711 Mouse Anti-Human CD19 | BD Biosciences | Cat. 563036 |
| | | RRID: AB_2737968 |
| BV786 Mouse Anti-Human CD279 | BD Biosciences | Cat. 563789 |
| | | RRID: AB_2738425 |
| PE Mouse Anti-Human CD206 | BD Biosciences | Cat. 566884 |
| | | RRID: AB_2869935 |
| PE-CF594 Mouse Anti-Human FoxP3 | BD Biosciences | Cat. 563955 |
| | | RRID: AB_2738507 |
| PE/Cyanine5 Anti-Human CD14 | BioLegend | Cat. 301864 |
| | | RRID: AB_2860767 |
| PE/Cyanine7 Anti-Human CD274 | BioLegend | Cat. 329718 |
| | | RRID: AB_2561687 |

TABLE 2
List of Mouse Antibodies

| ANTIBODIES | SOURCE | IDENTIFIER |
|---|---|---|
| APC Rat Anti-Mouse Ly-6G | BD Biosciences | Cat. 560599 |
| | | RRID: AB_1727560 |
| APC-R700 Rat Anti-Mouse CD25 | BD Biosciences | Cat. 565134 |
| | | RRID: AB_2744344 |
| APC/Cyanine7 Anti-Mouse I-A/I-E | BioLegend | Cat. 107628 |
| | | RRID: AB_1659252 |
| Alexa Fluor ® 488 Anti-Mouse Ki-67 | BioLegend | Cat. 652418 |
| | | RRID: AB_2564269 |
| BB700 Hamster Anti-Mouse CD103 | BD Biosciences | Cat. 748240 |
| | | RRID: AB_2872671 |
| BUV395 Rat Anti-Mouse CD45 | BD Biosciences | Cat. 564279 |
| | | RRID: AB_2651134 |
| BUV496 Rat Anti-Mouse CD4 | BD Biosciences | Cat. 612952 |
| | | RRID: AB_2813886 |
| BUV563 Rat Anti-CD11b | BD Biosciences | Cat. 741242 |
| | | RRID: AB_2870793 |
| BUV661 Rat Anti-Mouse F4/80 | BD Biosciences | Cat. 750643 |
| | | RRID: AB_2874771 |
| BUV737 Rat Anti-Mouse CD279 | BD Biosciences | Cat. 749306 |
| | | RRID: AB_2873680 |
| BUV805 Hamster Anti-Mouse CD3 | BD Biosciences | Cat. 749276 |
| | | RRID: AB_2873651 |
| BV421 ™ Anti-Mouse CD198 | BioLegend | Cat. 150305 |
| | | RRID: AB_2616650 |
| BV480 Rat Anti-Mouse CD8a | BD Biosciences | Cat. 566096 |
| | | RRID: AB_2739500 |
| Fixable Viability Stain 575V | BD Biosciences | Cat. 565694 |
| | | RRID: AB_2869702 |
| BV650 Hamster Anti-Mouse CD11c | BD Biosciences | Cat. 564079 |
| | | RRID: AB_2725779 |
| BV711 ™ Anti-Mouse Ly-6C | BioLegend | Cat. 128037 |
| | | RRID: AB_2562630 |
| BV786 Rat Anti-Mouse CD19 | BD Biosciences | Cat. 563333 |
| | | RRID: AB_2738141 |
| PE anti-mouse Granzyme B | ThermoFisher | Cat. 12-8898-82 |
| | Scientific | RRID: AB_10870787 |
| PE-CF594 Rat Anti-Mouse Foxp3 | BD Biosciences | Cat. 562466 |
| | | RRID: AB_11151905 |
| PE/Cyanine5 Anti-Mouse NK-1.1 | BioLegend | Cat. 108716 |
| | | RRID: AB_493590 |
| PE/Cyanine7 Anti-Mouse CD39 | BioLegend | Cat. 143806 |
| | | RRID: AB_2563394 |

### Bulk RNA-Sequencing and Bioinformatics Analysis

RNAs from anti-CCR8 antibody or irradiation treated, and from non-treated dissociated mouse tumor cells were extracted using RNeasy Plus Mini Kit (Qiagen) and a library was prepared using Stranded mRNA Prep Kit (Illumina). The generated library of each tumor sample was quantified by KAPA Library Quantification Kit (Roche) and normalized before pooling and loading onto the NextSeq 550 sequencer (Illumina). TITR, Treg cell, and CD8 T cell gene signatures are described by Meng et al. (2021) and Maguson et al., PNAS (2018). Gene set variation analysis (GSVA) was then performed to calculate the enrichment score of the summarized gene signatures. GSVA is a powerful and widely used statistical method that can be used to compare the activity of gene sets between two or more groups. In our case, the GSVA was used to compare the relatively cell enrichment in tumors under different treatment conditions. Gene Set Variation Analysis (GSVA) on those gene signatures was performed by GSVA (v1.42.0) R package (Hanzelmann et al., 2013).

### Statistical Analysis

GraphPad Prism 9.0 was used for all statistical analyses. Data are represented as mean±SD unless otherwise stated. One-way ANOVA with Dunnett's multiple comparison test was used for group comparisons. Results were considered significant when P<0.05.

### Results

The data described above indicate that Tregs are resistant to radiation therapy, while conventional CD4+ and CD8+ T cells decrease in quantity after radiation therapy, indicating a reduction in an anti-tumor inflammatory response. Furthermore, we discovered that CCR8 is exclusively expressed on TITR cells, and is therefore likely suitable as a therapeutic target, that may be used for depletion of TITR cells and induce an anti-tumor inflammatory response.

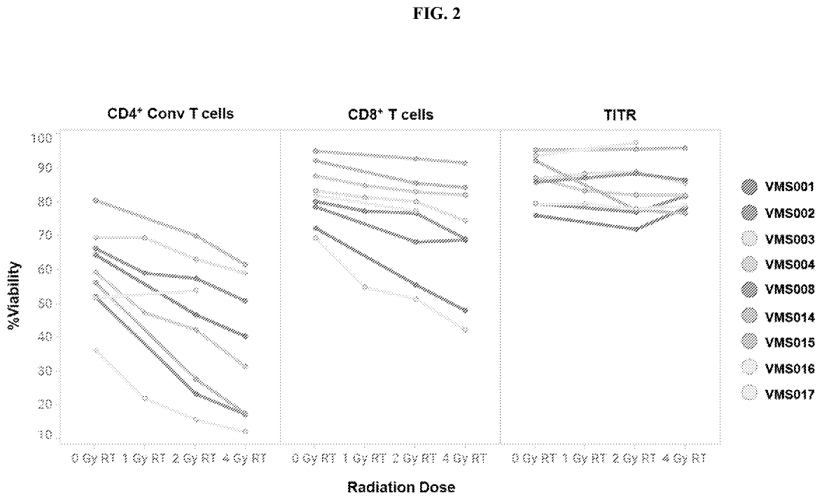

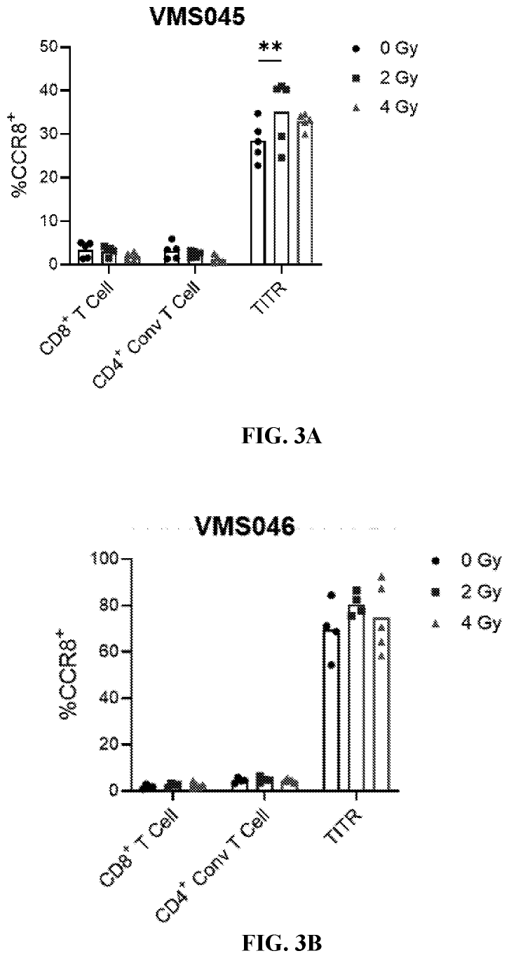

FIG. 2 shows percent viability of CD4⁺ conventional T cells, CD8⁺ T cells, and TITR cellsin ex vivo irradiated (0, 1, 2, and 4 Gy) human head and neck squamous cell carcinoma samples. FIGS. 3A-3D show percent CCR8 positive cells in different populations of T cells in human tumor samples following ex vivo radiation treatment at 0, 2, and 4 Gy. CCR8 expression is highly enriched on TITR.

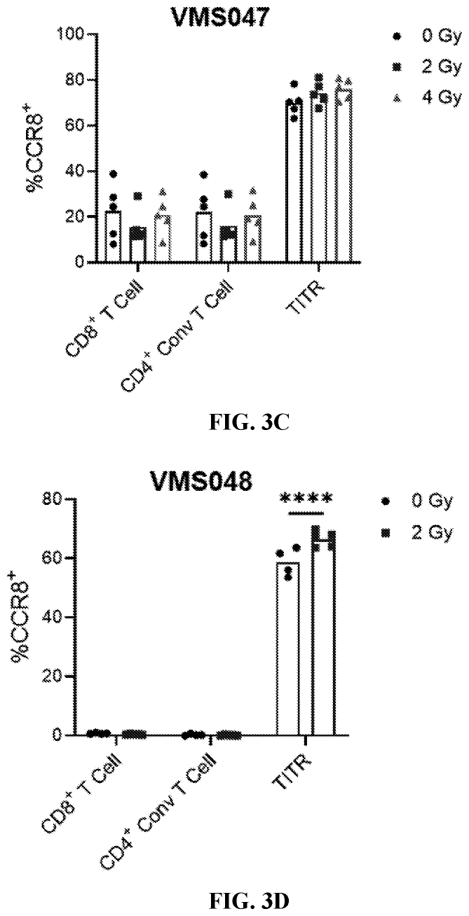

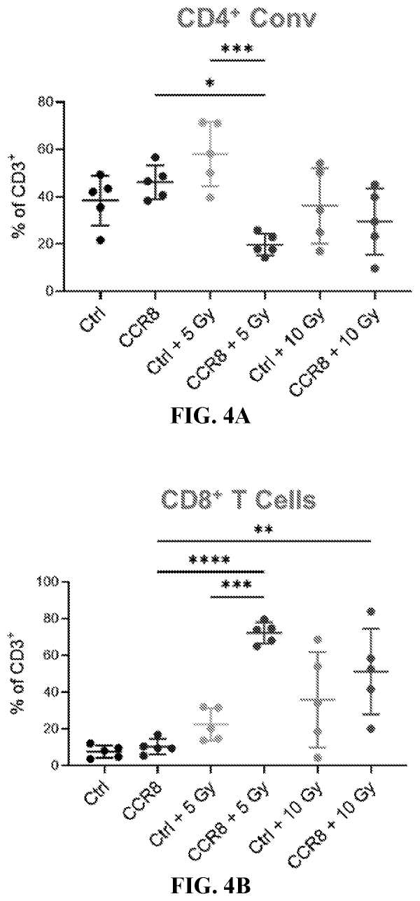

The combination of anti-CCR8 antibody therapy with radiation therapy also lead to an increased CD8+ population and decreased Treg cells over anti-CCR8 antibody therapy alone in the mouse tumor as measured by flow cytometry (FIGS. 4A-4C). FIG. 5 shows results of immunophenotyping by flow cytometry of excised and dissociated tumors from the syngeneic orthotopic breast cancer model. Combination of anti-CCR8 antibody+10 Gy RT lead to increased activation level of CD8⁺ T cells by T cell activation marker CD39 over anti-CCR8 antibody treatment alone.

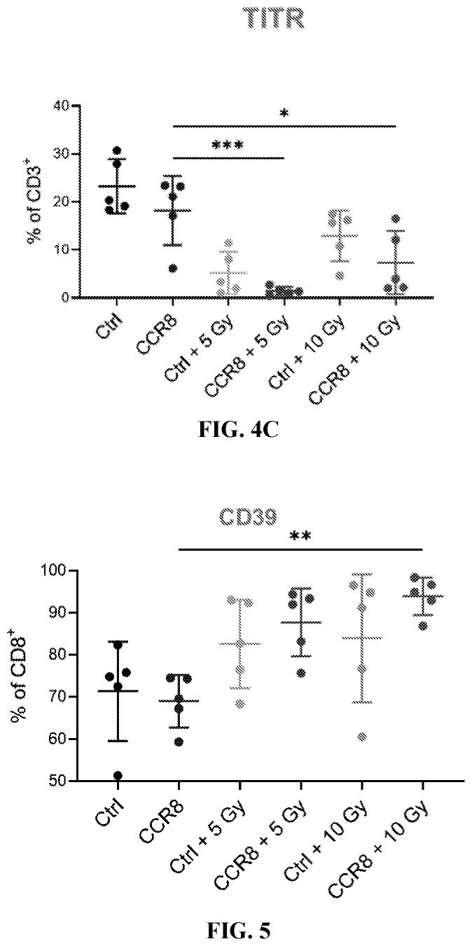

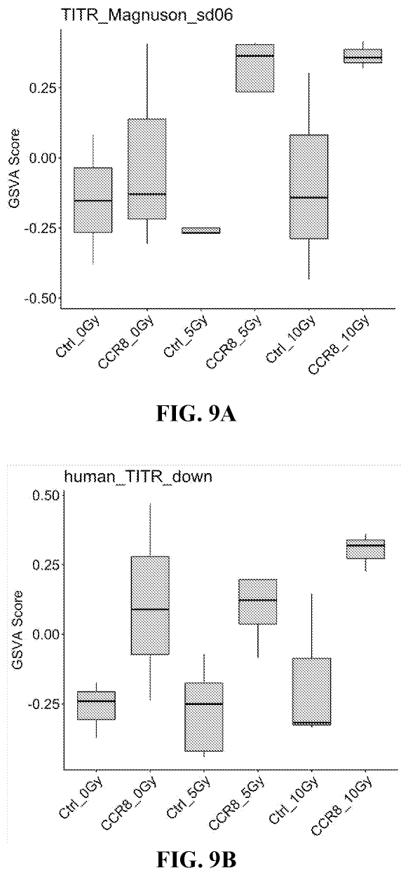

Moreover, the combination of the anti-CCR8 antibody and 10 Gy radiation therapy lead to improved efficacy over anti-CCR8 antibody alone in both in vivo models of breast cancer (FIG. 6) and lung cancer (FIG. 10). In both cancer models, anti-CCR8 antibody treatment alone had minimal efficacy as compared to control. In contrast, the combination of anti-CCR8 antibody with 10 Gy radiation therapy led to a significantly increased efficacy compared to either treatment (anti-CCR8 antibody or 10 Gy radiation) alone, in both cancer models.

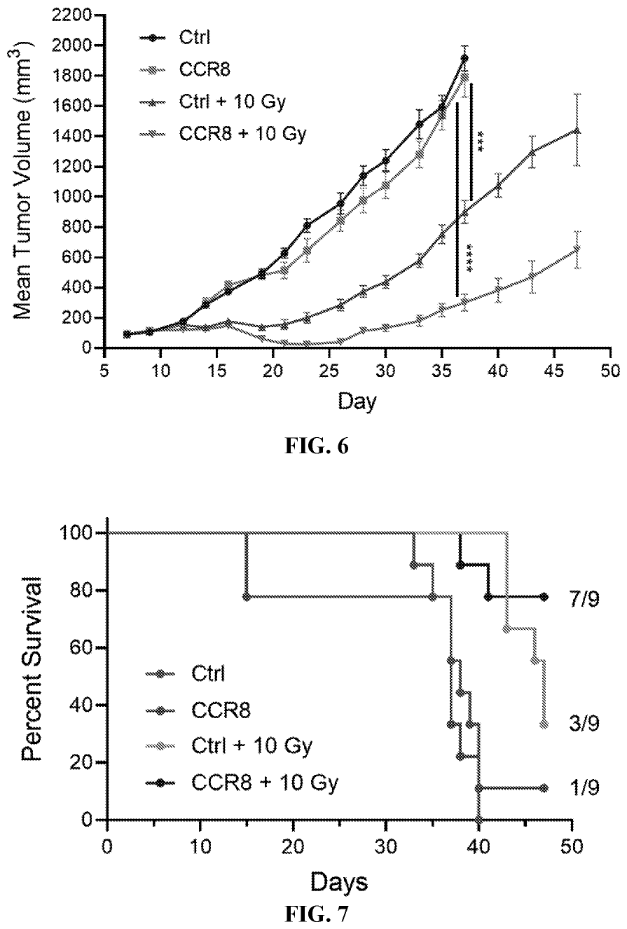

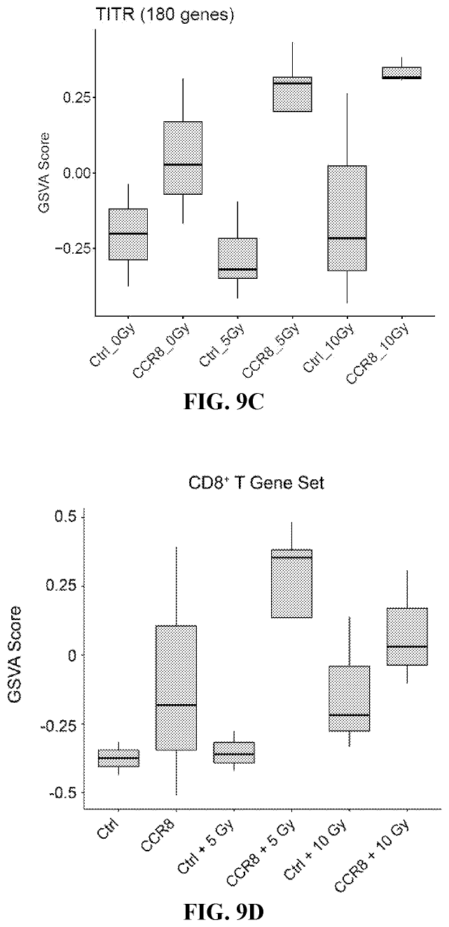

A Kaplan-Meier survival analysis of mice following control (Ctrl) treatment, anti-CCR8 antibody alone (CCR8), 10 Gy radiation alone (Ctrl+10 Gy), and anti-CCR8 antibody+10 Gy (CCR8+10 Gy) is shown, following combination treatment in vivo in both a model of breast cancer (FIG. 7) and lung cancer (FIG. 11). The combination of anti-CCR8 antibody with 10 Gy radiation therapy resulted in increased survival.

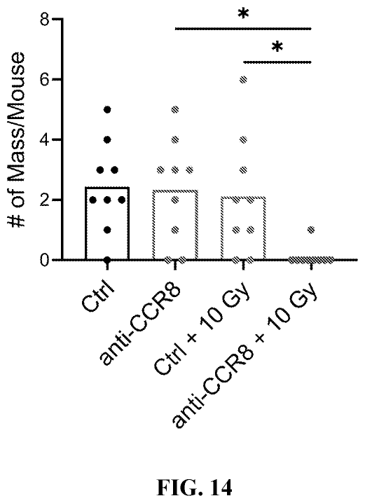

Treatment with the combination of anti-CCR8 and 10 Gy reduces cancer metastasis (FIG. 14). The number of tumor masses observed in various organs in mice treated with anti-CCR8 antibody and radiation therapy were significantly reduced compared with mice that received single-agent treatment alone, indicating higher metastatic control with the combination strategy.

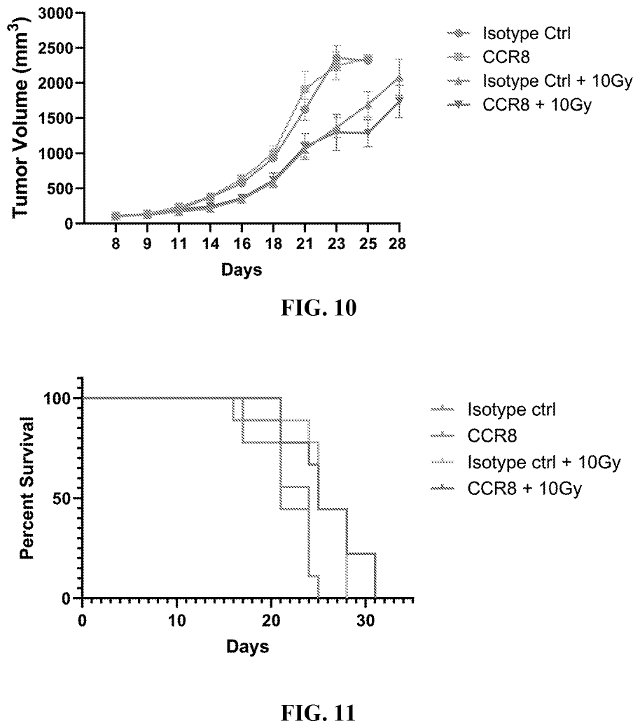

To test the effects of the combination treatment with additional therapeutic agents, a checkpoint inhibitor, anti-PD1 antibody, was used (PD1). Anti-CCR8 antibody (CCR8) or anti-CCR8 antibody and anti-PD1 antibody (CCR8+PD1) treatments alone do not have efficacy in the lung tumor model (FIG. 12). By contrast, a combination of anti-CCR8 antibody, PD1 antibody and radiation therapy (CCR8+PD1+10 Gy) demonstrated significantly reduced tumor volume compared with mice receiving CCR8, CCR8+PD1 or radiation therapy (Ctrl+10 Gy) treatments alone (FIG. 12).

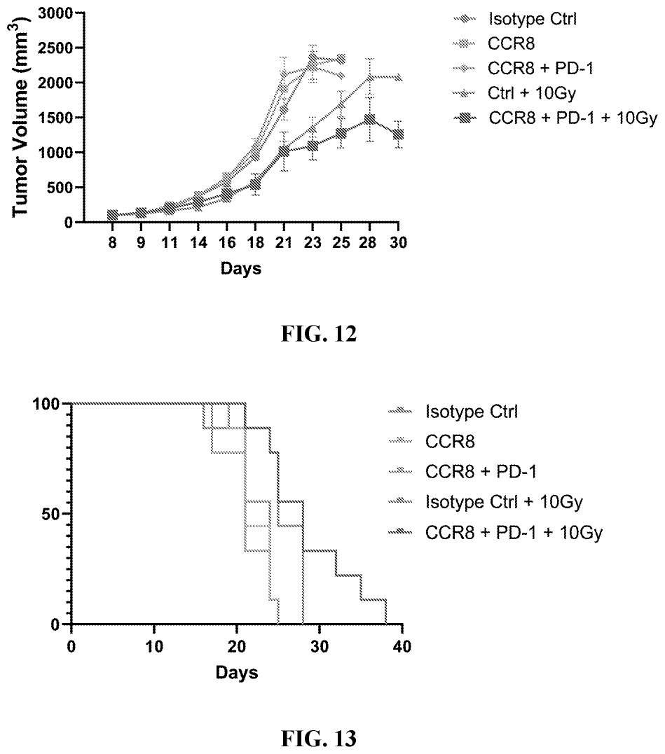

A Kaplan-Meier survival analysis of mice following treatment with control (Ctrl) treatment, anti-CCR8 antibody alone (CCR8), anti-CCR8 antibody and anti-PD1 antibody (CCR8+PD1), 10 Gy radiation alone (Ctrl+10 Gy), and anti-CCR8 antibody+ anti-PD1 antibody and radiation therapy (CCR8+PD1+10 Gy), is shown for an in vivo in a lung cancer model (FIG. 13). The combination of CCR8+PD1+10 Gy resulted in an improved survival compared with mice receiving CCR8 alone, CCR8+PD1 combination or Ctrl+10 Gy.

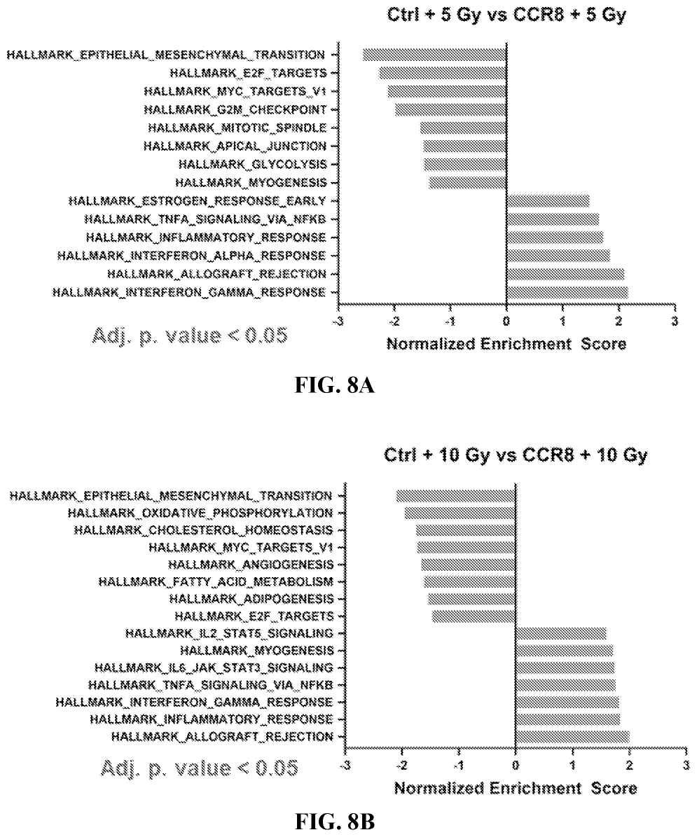

FIGS. 8A-8C show results of gene set enrichment analysis (GSEA) performed based on an RNA-sequencing study using dissociated tumor samples from the syngeneic orthotopic breast cancer model. Immune-associated gene sets are upregulated in anti-CCR8 antibody plus RT treated mouse tumor samples.

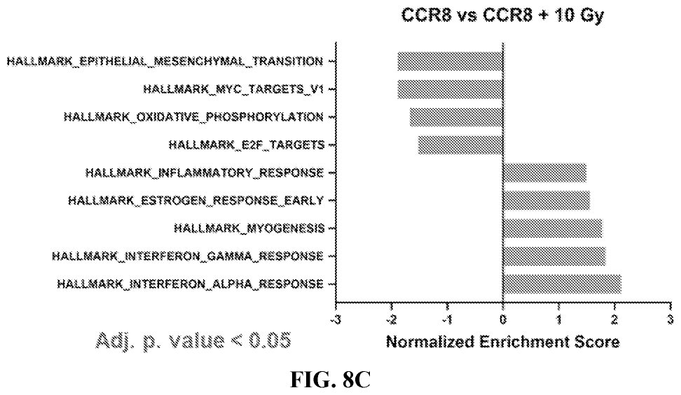

FIGS. 9A-9C show results of an RNA-sequencing study performed on dissociated tumor samples from the syngeneic orthotopic breast cancer model, indicating upregulation of a set of TITR down-regulated gene signatures (which indicates depletion of TITR cells) and upregulation of CD8⁺ T cell gene signatures in breast tumors after anti-CCR8 antibody treatment in combination with radiation therapy, consistent with the results from flow cytometry analysis. The results showed that a unique subset of TITR gene signatures are required to to identify the TITR cell population (e.g., an analysis of the 73 downregulated gene signatures). Shown are GVAS enrichment of different combinations of TITR gene signature (FIG. 9A-C) and CD8⁺ T cell gene signature (FIG. 9D) across control (Ctrl), anti-CCR8 antibody (CCR8), control+5 Gy, anti-CCR8 antibody+5 Gy, control+10 Gy, and anti-CCR8 antibody+10 Gy treatment groups.

In conclusion, this study suggests and outlines a novel way to enhance the effects of radiation therapy by combining radiation therapy with a TITR effector, such as anti-CCR8 immunotherapy. The results of the gene signature analysis studies suggest that a unique subset of TITR gene signatures can help to identify subjects with tumors with high levels of TITR cells, which is advantageous for stratifying a patent population to determine responders to a therapeutic treatment.

### Embodiments

Embodiment 1. A method of targeting tumor infiltrating regulatory T cell (TITR) cells in a subject, the method comprising administering to the subject a TITR effector and radiation therapy.

Embodiment 2. The method of embodiment 1, wherein the TITR effector is a CCR8 targeting agent.

Embodiment 3. The method of embodiment 1 or 2, wherein the TITR effector and the radiation therapy are administered concurrently.

Embodiment 4. The method of embodiment 1 or 2, wherein the TITR effector and the radiation therapy are administered sequentially.

Embodiment 5. The method of any one of embodiments 1-4, wherein the TITR effector, the radiation therapy, or both are administered repeatedly.

Embodiment 6. The method of any one of embodiments 1-5, wherein the subject is afflicted with cancer.

Embodiment 7. The method of embodiment 6, wherein the cancer is breast cancer, lung cancer, head and neck cancer, liver cancer, pancreatic cancer, brain cancer, colorectal cancer, or prostate cancer.

Embodiment 8. The method of any one of embodiments 1-7, wherein the cancer has previously been treated with radiotherapy and/or the TITR effector.

Embodiment 9. The method of any one of embodiments 1-8, wherein the cancer is resistant to radiotherapy and/or the TITR effector.

Embodiment 10. The method of any one of embodiments 1-9, wherein the radiotherapy is electron radiation, proton radiation, photon radiation, a radiopharmaceutical, or brachytherapy.

Embodiment 11. The method of any one of embodiments 1-10, wherein the radiotherapy is ultra-high dose rate (FLASH) radiotherapy.

Embodiment 12. The method of any one of embodiments 1-11, wherein the method results in a reduction in TITR number in the cancer relative to the TITR number prior to the administration.

Embodiment 13. A method of treating cancer in a subject, the method comprising (a) obtaining a sample from the cancer and a normal tissue sample; (b) quantifying the expression level of at least 3, at least 4, at least 5, at least 6, at least 7, at least 8, at least 9, at least 10, at least 11, at least 12, at least 13, at least 14, at least 15, at least 16, at least 17, at least 18, at least 19, at least 20 genes, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110 genes, at least 115 genes, at least 120 genes, at least 125, at least 130 genes, at least 135 genes, at least 140 genes, at least 145, at least 150 genes, at least 155 genes, at least 160 genes, at least 165, at least 170 genes, at least 175 genes, or at least 180 genes and (c) administering to the subject a combination therapy comprising a TITR effector and radiotherapy if a TITR gene signature is identified in the tumor compared to normal tissue using an over-representation/enrichment method;

wherein the genes are selected from CCR8, CD80, CSF2RB, ENTPD1, EBI3, TNFRSF8, TNFRSF18, DUSP4, SDC4, LAPTM4B, TNFRSF4, SEC14L1, TNFRSF9, IRAK2, FNDC3B, IKZF4, IL1R2, NINJ1, SYNGR2, TNIP2, SSH1, ARHGEF12, GCNT1, NCF4, MAPKAPK2, RHBDD2, TRAF3, IRF5, ACOT9, HIVEP3, RFK, UEVLD, BCL2L1, FAM129A, BATF, RHOC, ZNRF1, PMAIP1, PLAGL2, MRPS6, IL1RL1, CREB3L2, CPD, PICALM, CCR5, CTNNA1, GALC, TTC39C, CYFIP1, STAT1, FURIN, TRAF1, GBP5, HIF1A, UBE2L6, PTPRJ, TNFRSF1B, REM, CAPN2, TRIM16, CD74, MAP2K3, TIGIT CTSH, TAP1, SKAP2, ITGAE, JAK3, CAPG, OPTN, NDRG1, DUSP16, CD274, ICOS, GLRX, SAMSN1, ANXA4, MAPKAPK3, KATNAL1, CDKN1A, SNX9. PFKL, ABI2, ATP2B4, GM2A, PGM2, RIPK3, ISG15, NAMPT, MVP, GOT1, MXD1, TMBIM1, RORA, PCYT1A, N4BP1, LAMP2, TMEM159, OSBPL3, VIM, ATF3, CXCR3, ALDOC, PLP2, FAM126A, OXSR1, GRN, APAF1, ANK3, ANKRD55, ANXA2R, ATM, BAZ2B, CCR7, CD200, CECR1, CLUHP3, CXCR5, DPH5, EPHA1, EPHA4, FAM174B, FAM65B, FASTKD2, FOXP1, GIMAP4, GIMAP5, GIMAP6, GIMAP7, GIMAP8, GZMK, HIVEP2, IL10, INADL, INPP4B, IQGAP2, KCNA3, KIAA1009, KIAA2026, KLF3, KLRG1, LDLRAP1, LEF1, LMO7, LOC100130231, LPAR6, LRIG1, LY9, MAP3K3, MS4A1, NAP1L2, OBSCN, PAR-SN, PLAC8, PLAG1, PLEK, PLK2, PLXDC1, PRKCQ-AS1, RNF214, SERINC5, SESN1, SLAMF7, SLC16A7, SLC9A6, SMAD3, SPINT2, SSBP2, STXBP5, TCF7, TENM1, TMEM243, TRAT1, TTC21B, TXK, ZBTB10, ZNF248, ZNF480, ZNF737, ZNF780B, and ZNF91.

Embodiment 14. A method for selecting a patient for treatment with a combination therapy comprising a TITR effector and radiation therapy, the method comprising (a) obtaining a sample from the cancer and a normal tissue sample; (b) quantifying the expression level of at least 3, at least 4, at least 5, at least 6, at least 7, at least 8, at least 9, at least 10, at least 11, at least 12, at least 13, at least 14, at least 15, at least 16, at least 17, at least 18, at least 19, at least 20 genes, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110 genes, at least 115 genes, at least 120 genes, at least 125, at least 130 genes, at least 135 genes, at least 140 genes, at least 145, at least 150 genes, at least 155 genes, at least 160 genes, at least 165, at least 170 genes, at least 175 genes, or at least 180 genes; and (c) selecting the patient for treatment if a TITR gene signature is identified in the tumor compared to normal tissue using an over-representation/enrichment method;

wherein the genes are selected from CCR8, CD80, CSF2RB, ENTPD1, EBI3, TNFRSF8, TNFRSF18, DUSP4, SDC4, LAPTM4B, TNFRSF4, SEC14L1, TNFRSF9, IRAK2, FNDC3B, IKZF4, IL1R2, NINJ1, SYNGR2, TNIP2, SSH1, ARHGEF12, GCNT1, NCF4, MAPKAPK2, RHBDD2, TRAF3, IRF5, ACOT9, HIVEP3, RFK, UEVLD, BCL2L1, FAM129A, BATF, RHOC, ZNRF1, PMAIP1, PLAGL2, MRPS6, IL1RL1, CREB3L2, CPD, PICALM, CCR5, CTNNA1, GALC, TTC39C, CYFIP1, STAT1, FURIN, TRAF1, GBP5, HIF1A, UBE2L6, PTPRJ, TNFRSF1B, REM, CAPN2, TRIM16, CD74, MAP2K3, TIGIT, CTSH, TAP1, SKAP2, ITGAE, JAK3, CAPG, OPTN, NDRG1, DUSP16, CD274, ICOS, GLRX, SAMSN1, ANXA4, MAPKAPK3, KATNAL1, CDKN1A, SNX9. PFKL, ABI2, ATP2B4, GM2A, PGM2, RIPK3, ISG15, NAMPT, MVP, GOT1, MXD1, TMBIM1, RORA, PCYT1A, N4BP1, LAMP2, TMEM159, OSBPL3, VIM, ATF3, CXCR3, ALDOC, PLP2, FAM126A, OXSR1, GRN, APAF1, ANK3, ANKRD55, ANXA2R, ATM, BAZ2B, CCR7, CD200, CECR1, CLUHP3, CXCR5, DPH5, EPHA1, EPHA4, FAM174B, FAM65B, FASTKD2, FOXP1, GIMAP4, GIMAP5, GIMAP6, GIMAP7, GIMAP8, GZMK, HIVEP2, IL10, INADL, INPP4B, IQGAP2, KCNA3, KIAA1009, KIAA2026, KLF3, KLRG1, LDLRAP1, LEF1, LMO7, LOC100130231, LPAR6, LRIG1, LY9, MAP3K3, MS4A1, NAP1L2, OBSCN, PAR-SN, PLAC8, PLAG1, PLEK, PLK2, PLXDC1, PRKCQ-AS1, RNF214, SERINC5, SESN1, SLAMF7, SLC16A7, SLC9A6, SMAD3, SPINT2, SSBP2, STXBP5, TCF7, TENM1, TMEM243, TRAT1, TTC21B, TXK, ZBTB10, ZNF248, ZNF480, ZNF737, ZNF780B, and ZNF91.

Embodiment 15. The method of any one of embodiments 13 or 14, wherein the TITR effector is a CCR8 targeting agent.

Embodiment 16. The method of any one of embodiments 13-15, wherein the radiotherapy is electron radiation, proton radiation, or photon radiation.

Embodiment 17. The method of any one of embodiments 13-16, wherein the radiotherapy is ultra-high dose rate (FLASH) radiotherapy.

Embodiment 18. The method of any one of embodiments 13-17, wherein the cancer is breast cancer, lung cancer, head and neck cancer, liver cancer, pancreatic cancer, brain cancer, colorectal cancer, or prostate cancer.

Embodiment 19. Use of a plurality of agents specific to quantifying the expression level of at least 3, at least 4, at least 5, at least 6, at least 7, at least 8, at least 9, at least 10, at least 11, at least 12, at least 13, at least 14, at least 15, at least 16, at least 17, at least 18, at least 19, at least 20 genes, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110 genes, at least 115 genes, at least 120 genes, at least 125, at least 130 genes, at least 135 genes, at least 140 genes, at least 145, at least 150 genes, at least 155 genes, at least 160 genes, at least 165, at least 170 genes, at least 175 genes, or at least 180 genes; and (c) producing a report, wherein the report identifies whether the cancer has a TITR gene signature using an over-representation/enrichment method;

wherein the genes are selected from CCR8, CD80, CSF2RB, ENTPD1, EBI3, TNFRSF8, TNFRSF18, DUSP4, SDC4, LAPTM4B, TNFRSF4, SEC14L1, TNFRSF9, IRAK2, FNDC3B, IKZF4, IL1R2, NINJ1, SYNGR2, TNIP2, SSH1, ARHGEF12, GCNT1, NCF4, MAPKAPK2, RHBDD2, TRAF3, IRF5, ACOT9, HIVEP3, RFK, UEVLD, BCL2L1, FAM129A, BATF, RHOC, ZNRF1, PMAIP1, PLAGL2, MRPS6, IL1RL1, CREB3L2, CPD, PICALM, CCR5, CTNNA1, GALC, TTC39C, CYFIP1, STAT1, FURIN, TRAF1, GBP5, HIF1A, UBE2L6, PTPRJ, TNFRSF1B, REM, CAPN2, TRIM16, CD74, MAP2K3, TIGIT, CTSH, TAP1, SKAP2, ITGAE, JAK3, CAPG, OPTN, NDRG1, DUSP16, CD274, ICOS, GLRX, SAMSN1, ANXA4, MAPKAPK3, KATNAL1, CDKN1A, SNX9. PFKL, ABI2, ATP2B4, GM2A, PGM2, RIPK3, ISG15, NAMPT, MVP, GOT1, MXD1, TMBIM1, RORA, PCYT1A, N4BP1, LAMP2, TMEM159, OSBPL3, VIM, ATF3, CXCR3, ALDOC, PLP2, FAM126A, OXSR1, GRN, APAF1, ANK3, ANKRD55, ANXA2R, ATM, BAZ2B, CCR7, CD200, CECR1, CLUHP3, CXCR5, DPH5, EPHA1, EPHA4, FAM174B, FAM65B, FASTKD2, FOXP1, GIMAP4, GIMAP5, GIMAP6, GIMAP7, GIMAP8, GZMK, HIVEP2, IL10, INADL, INPP4B, IQGAP2, KCNA3, KIAA1009, KIAA2026, KLF3, KLRG1, LDLRAP1, LEF1, LMO7, LOC100130231, LPAR6, LRIG1, LY9, MAP3K3, MS4A1, NAP1L2, OBSCN, PAR-SN, PLAC8, PLAG1, PLEK, PLK2, PLXDC1, PRKCQ-AS1, RNF214, SERINC5, SESN1, SLAMF7, SLC16A7, SLC9A6, SMAD3, SPINT2, SSBP2, STXBP5, TCF7, TENM1, TMEM243, TRAT1, TTC21B, TXK, ZBTB10, ZNF248, ZNF480, ZNF737, ZNF780B, and ZNF9L.

Embodiment 20. The use of embodiment 19, wherein the cancer is breast cancer, lung cancer, head and neck cancer, liver cancer, pancreatic cancer, brain cancer, colorectal cancer, or prostate cancer.

What is claimed is:

1. A method of decreasing a population of tumor infiltrating regulatory T cell (TITR) cells in a subject, the method comprising administering to the subject a TITR effector and a radiation therapy.

2. The method of claim 1, wherein the TITR effector is a CCR8 targeting agent.

3. The method of claim 1 or 2, wherein the TITR effector and the radiation therapy are administered concurrently.

4. The method of any one of claims 1-3, wherein the TITR effector, the radiation therapy, or both are administered at least one time, at least two times, at least three times, at least four times or at least five times.

5. The method of any one of claims 1-4, comprising administering a checkpoint inhibitor selected from a PD-1 inhibitor, a PD-L1 inhibitor, a CTLA-4 inhibitor, a TIM-3 inhibitor, a CEACAM inhibitor, a LAG-3 inhibitor, a VISTA inhibitor, a BTLA inhibitor, a TIGIT inhibitor, a LAIR1 inhibitor, a CD160 inhibitor, a 2B4 inhibitor or a TGFR beta inhibitor.

6. The method of claim 5, wherein the checkpoint inhibitor is a PD-1 inhibitor.

7. The method of any one of claims 5-6, wherein the CCR8 targeting agent, the radiation therapy and/or the checkpoint inhibitor are administered concurrently or sequentially.

8. The method of any one of claims 5-7, wherein the CCR8 targeting agent, the radiation therapy and/or the checkpoint inhibitor are administered at least one time, at least two times, at least three times, at least four times or at least five times.

9. The method of any one of claims 1-8, wherein the subject has a cancer.

10. The method of claim 9, wherein the cancer is breast cancer, lung cancer, head and neck cancer, liver cancer, pancreatic cancer, brain cancer, colorectal cancer, or prostate cancer.

11. The method of any one of claims 9-10, wherein the cancer is resistant to the radiation therapy and/or the TITR effector.

12. The method of any one of claims 1-11, wherein the subject has previously been treated with the radiation therapy and/or the TITR effector.

13. The method of any one of claims 1-12, wherein the radiation therapy is electron radiation, proton radiation, photon radiation, a radiopharmaceutical, or brachytherapy.

14. The method of any one of claims 1-13, wherein the radiation therapy is ultra-high dose rate (FLASH) radiotherapy.

15. The method of any one of claims 1-14, wherein the method results in a decrease of about 5%, about 10%, about 20%, about 30%, about 40%, about 50%, about 60%, about 70%, about 80%, or about 90% of the number of TITR cells in the subject relative to the number of TITR cells in the subject prior to the administration of the TITR effector and the radiation therapy.

16. A method of treating cancer in a subject, the method comprising:
(a) obtaining a tumor sample from the cancer and a normal tissue sample;
(b) quantifying the expression level of at least 3, at least 4, at least 5, at least 6, at least 7, at least 8, at least 9, at least 10, at least 11, at least 12, at least 13, at least 14, at least 15, at least 16, at least 17, at least 18, at least 19, at least 20 genes, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110 genes, at least 115 genes, at least 120 genes, at least 125, at least 130 genes, at least 135 genes, at least 140 genes, at least 145, at least 150 genes, at least 155 genes, at least 160 genes, at least 165, at least 170 genes, at least 175 genes, or at least 180 genes; and
(c) administering to the subject a combination therapy comprising a TITR effector and a radiotherapy if a TITR gene signature is identified in the tumor sample compared to the normal tissue sample using an over-representation/enrichment method;
wherein the genes are selected from CCR8, CD80, CSF2RB, ENTPD1, EBI3, TNFRSF8, TNFRSF18, DUSP4, SDC4, LAPTM4B, TNFRSF4, SEC14L1, TNFRSF9, IRAK2, FNDC3B, IKZF4, IL1R2, NINJ1, SYNGR2, TNIP2, SSH1, ARHGEF12, GCNT1, NCF4, MAPKAPK2, RHBDD2, TRAF3, IRF5, ACOT9, HIVEP3, RFK, UEVLD, BCL2L1, FAM129A, BATF, RHOC, ZNRF1, PMAIP1, PLAGL2, MRPS6, IL1RL1, CREB3L2, CPD, PICALM, CCR5, CTNNA1, GALC, TTC39C, CYFIP1, STAT1, FURIN, TRAF1, GBP5, HIF1A, UBE2L6, PTPRJ, TNFRSF1B, REM, CAPN2, TRIM16, CD74, MAP2K3, TIGIT, CTSH, TAP1, SKAP2, ITGAE, JAK3, CAPG, OPTN, NDRG1, DUSP16, CD274, ICOS, GLRX, SAMSN1, ANXA4, MAPKAPK3, KATNAL1, CDKN1A, SNX9. PFKL, ABI2, ATP2B4, GM2A, PGM2, RIPK3, ISG15, NAMPT, MVP, GOT1, MXD1, TMBIM1, RORA, PCYT1A, N4BP1, LAMP2, TMEM159, OSBPL3, VIM, ATF3, CXCR3, ALDOC, PLP2, FAM126A, OXSR1, GRN, APAF1, ANK3, ANKRD55, ANXA2R, ATM, BAZ2B, CCR7, CD200, CECR1, CLUHP3, CXCR5, DPH5, EPHA1, EPHA4, FAM174B, FAM65B, FASTKD2, FOXP1, GIMAP4, GIMAP5, GIMAP6, GIMAP7, GIMAP8, GZMK, HIVEP2, IL10, INADL, INPP4B, IQGAP2, KCNA3, KIAA1009, KIAA2026, KLF3, KLRG1, LDLRAP1, LEF1, LMO7, LOC100130231, LPAR6, LRIG1, LY9, MAP3K3, MS4A1, NAP1L2, OBSCN, PAR-SN, PLAC8, PLAG1, PLEK, PLK2, PLXDC1, PRKCQ-AS1, RNF214, SERINC5, SESN1, SLAMF7, SLC16A7, SLC9A6, SMAD3, SPINT2, SSBP2, STXBP5, TCF7, TENM1, TMEM243, TRAT1, TTC21B, TXK, ZBTB10, ZNF248, ZNF480, ZNF737, ZNF780B, and ZNF91.

17. A method for selecting a subject having a cancer for treatment with a combination therapy comprising a TITR effector and a radiation therapy, the method comprising:
(a) obtaining a tumor sample from the cancer and a normal tissue sample;
(b) quantifying the expression level of at least 3, at least 4, at least 5, at least 6, at least 7, at least 8, at least 9, at least 10, at least 11, at least 12, at least 13, at least 14, at least 15, at least 16, at least 17, at least 18, at least 19, at least 20 genes, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110 genes, at least 115 genes, at least 120 genes, at least 125, at least 130 genes, at least 135 genes, at least 140 genes, at least 145, at least 150 genes, at least 155 genes, at least 160 genes, at least 165, at least 170 genes, at least 175 genes, or at least 180 genes; and
(c) selecting the subject for treatment if a TITR gene signature is identified in the tumor sample compared to the normal tissue sample using an over-representation/enrichment method;
wherein the genes are selected from CCR8, CD80, CSF2RB, ENTPD1, EBI3, TNFRSF8, TNFRSF18, DUSP4, SDC4, LAPTM4B, TNFRSF4, SEC14L1, TNFRSF9, IRAK2, FNDC3B, IKZF4, IL1R2, NINJ1, SYNGR2, TNIP2, SSH1, ARHGEF12, GCNT1, NCF4, MAPKAPK2, RHBDD2, TRAF3, IRF5, ACOT9, HIVEP3, RFK, UEVLD, BCL2L1, FAM129A, BATF, RHOC, ZNRF1, PMAIP1, PLAGL2, MRPS6, IL1RL1, CREB3L2, CPD, PICALM, CCR5, CTNNA1, GALC, TTC39C, CYFIP1, STAT1, FURIN, TRAF1, GBP5, HIF1A, UBE2L6, PTPRJ, TNFRSF1B, REM, CAPN2, TRIM16, CD74, MAP2K3, TIGIT CTSH, TAP1, SKAP2, ITGAE, JAK3, CAPG, OPTN, NDRG1, DUSP16, CD274, ICOS, GLRX, SAMSN1, ANXA4, MAPKAPK3, KATNAL1, CDKN1A, SNX9. PFKL, ABI2, ATP2B4, GM2A, PGM2, RIPK3, ISG15, NAMPT, MVP, GOT1, MXD1, TMBIM1, RORA, PCYT1A, N4BP1, LAMP2, TMEM159, OSBPL3, VIM, ATF3, CXCR3, ALDOC, PLP2, FAM126A, OXSR1, GRN, APAF1, ANK3, ANKRD55, ANXA2R, ATM, BAZ2B, CCR7, CD200, CECR1, CLUHP3, CXCR5, DPH5, EPHA1, EPHA4, FAM174B, FAM65B, FASTKD2, FOXP1, GIMAP4, GIMAP5, GIMAP6, GIMAP7, GIMAP8, GZMK, HIVEP2, IL10, INADL, INPP4B, IQGAP2, KCNA3, KIAA1009, KIAA2026, KLF3, KLRG1, LDLRAP1, LEF1, LMO7, LOC100130231, LPAR6, LRIG1, LY9, MAP3K3, MS4A1, NAP1L2, OBSCN, PAR-SN, PLAC8, PLAG1, PLEK, PLK2, PLXDC1, PRKCQ-AS1, RNF214, SERINC5, SESN1, SLAMF7, SLC16A7, SLC9A6, SMAD3, SPINT2, SSBP2, STXBP5, TCF7, TENM1, TMEM243, TRAT1, TTC21B, TXK, ZBTB10, ZNF248, ZNF480, ZNF737, ZNF780B, and ZNF91.

18. The method of any one of claims 16-17, wherein the TITR effector is a CCR8 targeting agent.

19. The method of any one of claims 16-18, wherein the radiation therapy is electron radiation, proton radiation, or photon radiation.

20. The method of any one of claims 16-19, wherein the radiation therapy is ultra-high dose rate (FLASH) radiotherapy.

21. The method of any one of claims 16-20, wherein the cancer is breast cancer, lung cancer, head and neck cancer, liver cancer, pancreatic cancer, brain cancer, colorectal cancer, or prostate cancer.

22. Use of a plurality of agents for quantifying the expression level of at least 3, at least 4, at least 5, at least 6, at least 7, at least 8, at least 9, at least 10, at least 11, at least 12, at least 13, at least 14, at least 15, at least 16, at least 17, at least 18, at least 19, at least 20 genes, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110 genes, at least 115 genes, at least 120 genes, at least 125, at least 130 genes, at least 135 genes, at least 140 genes, at least 145, at least 150 genes, at least 155 genes, at least 160 genes, at least 165, at least 170 genes, at least 175 genes, or at least 180 genes, in a cancer; and producing a report, wherein the report identifies whether the cancer has a TITR gene signature using an over-representation/enrichment method; and
wherein the genes are selected from CCR8, CD80, CSF2RB, ENTPD1, EBI3, TNFRSF8, TNFRSF18, DUSP4, SDC4, LAPTM4B, TNFRSF4, SEC14L1, TNFRSF9, IRAK2, FNDC3B, IKZF4, IL1R2, NINJ1, SYNGR2, TNIP2, SSH1, ARHGEF12, GCNT1, NCF4, MAPKAPK2, RHBDD2, TRAF3, IRF5, ACOT9, HIVEP3, RFK, UEVLD, BCL2L1, FAM129A, BATF, RHOC, ZNRF1, PMAIP1, PLAGL2, MRPS6, IL1RL1, CREB3L2, CPD, PICALM, CCR5, CTNNA1, GALC, TTC39C, CYFIP1, STAT1, FURIN, TRAF1, GBP5, HIF1A, UBE2L6, PTPRJ, TNFRSF1B, REM, CAPN2, TRIM16, CD74, MAP2K3, TIGIT, CTSH, TAP1, SKAP2, ITGAE, JAK3, CAPG, OPTN, NDRG1, DUSP16, CD274, ICOS, GLRX, SAMSN1, ANXA4, MAPKAPK3, KATNAL1, CDKN1A, SNX9. PFKL, ABI2, ATP2B4, GM2A, PGM2, RIPK3, ISG15, NAMPT, MVP, GOT1, MXD1, TMBIM1, RORA, PCYT1A, N4BP1, LAMP2, TMEM159, OSBPL3, VIM, ATF3, CXCR3, ALDOC, PLP2, FAM126A, OXSR1, GRN, APAF1, ANK3, ANKRD55, ANXA2R, ATM, BAZ2B, CCR7, CD200, CECR1, CLUHP3, CXCR5, DPH5, EPHA1, EPHA4, FAM174B, FAM65B, FASTKD2, FOXP1, GIMAP4, GIMAP5, GIMAP6, GIMAP7, GIMAP8, GZMK, HIVEP2, IL10, INADL, INPP4B, IQGAP2, KCNA3, KIAA1009, KIAA2026, KLF3, KLRG1, LDLRAP1, LEF1, LMO7, LOC100130231, LPAR6, LRIG1, LY9, MAP3K3, MS4A1, NAP1L2, OBSCN, PAR-SN, PLAC8, PLAG1, PLEK, PLK2, PLXDC1, PRKCQ-AS1, RNF214, SERINC5, SESN1, SLAMF7, SLC16A7, SLC9A6, SMAD3, SPINT2, SSBP2, STXBP5, TCF7, TENM1, TMEM243, TRAT1, TTC21B, TXK, ZBTB10, ZNF248, ZNF480, ZNF737, ZNF780B, and ZNF9L.

23. The use of claim 22, wherein the cancer is breast cancer, lung cancer, head and neck cancer, liver cancer, pancreatic cancer, brain cancer, colorectal cancer, or prostate cancer.
